(19) United States
(12) Patent Application Publication
(10) Pub. No.: US 2026/0226036 A1
(43) Pub. Date: Aug. 6, 2026

(54) THIOUREA ANTIOXCOMPOUNDS WITH NEUROPROTECTIVE ACTIVITY

(71) Applicant: UNIVERSITY OF NORTH TEXAS HEALTH SCIENCE CENTER, Fort Worth, TX (US)

(72) Inventors: Suchismita ACHARYA, Euless, TX (US); Ritu SHETTY, Fort Worth, TX (US)

(73) Assignee: UNIVERSITY OF NORTH TEXAS HEALTH SCIENCE CENTER, Fort Worth, TX (US)

(21) Appl. No.: 19/150,622

(22) PCT Filed: Jan. 24, 2024
(86) PCT No.: PCT/US2024/012694

§ 371 Date: Jul. 24, 2025

Related U.S. Application Data
(60) Provisional application No. 63/481,491, filed on Jan. 25, 2023.

(51) Int. Cl. C07D 409/12; A61K 31/4155; A61K 31/4468; A61K 31/4535; A61P 25/28; A61P 25/30; A61P 39/06; C07D 211/94; C07D 335/02

(52) U.S. Cl. CPC C07D 409/12; A61K 31/4155; A61K 31/4468; A61K 31/4535; A61P 25/28; A61P 25/30; A61P 39/06; C07D 211/94; C07D 335/02

(57) ABSTRACT

Provided herein are compounds of the formula wherein the variables are as defined herein. Pharmaceutical compositions of the compounds are also provided. In some aspects, the compounds or compositions of the present disclosure may be used for the treatment of diseases or disorders, such as an addiction or neurodegenerative disease including Alzheimer's disease or Parkinson's disease.

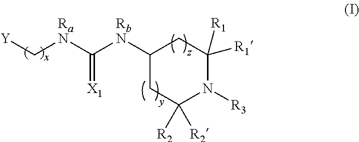

This application claims the benefit of priority to U.S. Provisional Application No. 63/481,491, filed on Jan. 25, 2023, the entire contents of which are hereby incorporated by reference.

### BACKGROUND OF THE DISCLOSURE

### I. Field of the Disclosure

The present disclosure relates generally to the fields of biology, chemistry, and medicine. More particularly, it concerns compounds, compositions and methods for the treatment and prevention of diseases and disorders, such as an addiction and/or neurogenerative diseases.

### II. Description of Related Art

Drug addiction or SUD is characterized by mental, physical, and behavioral symptoms as classified in Diagnostic and Statistical Manual of Mental Disorders (DSM-V), where an individual becomes increasingly dependent on one or more substance/s. According to DSM-V, SUD is a combination of substance abuse disorder and substance dependence disorder. The National Survey on Drug Use and Health (NSUDH) 2018 reported that 19.3 million people aged 18 or older struggled with SUD (Substance Abuse and Mental Health Services Administration, SAMHSA). Cocaine, as a pharmacological agent, inhibits reuptake of monoamines such as dopamine (DA), norepinephrine (NE), and serotonin (SERT) in the presynaptic terminals and is considered a psychomotor stimulant both in the central nervous system (CNS) and peripheral nervous system (PNS). Experts in the addiction field are predicting that methamphetamine (METH) abuse will become the next drug crisis (Nielsen et al., 2012), and indeed, mortality has doubled in the last 10 years. There are no FDA approved medications for treatment of methamphetamine use disorder (MUD), and the available supportive treatments have either failed or have marginal effects. Consequently, there is a dire need for novel therapies that target the underlying mechanisms that drive this disorder.

Oxidative-stress induced cell death is involved in the pathology of psychostimulant (cocaine and methamphetamine) addiction neuropathies (Nielsen et al., 2012). These conditions potentially cause neuronal and functional changes changes via different mechanisms: epigenetic alterations (DNA hypomethylation) and reactive-oxygen species (ROS) accumulation. Current medications for the treatment of psychostimulant-induced addiction neuropathies are largely ineffective due to the high rate of relapse and marginal alterations of addictive dependency.

Overstimulation of the sympathetic nervous system (SNS) with the engagement of the central nervous system (CNS) produces ROS (Slivka et al., 1985; Graham, 1978). Increased ROS production, including superoxide (O₂⁻) and peroxyl radical (OH) from psychostimulant exposure, results in covalent modifications of the active site of methionine synthase (MS) by reaction with ·OH radical. MS is a key enzyme that converts homocysteine to methionine in presence of Vit B₁₂ and 5-methyl tetrahydrofolate and promotes DNA methylation. Deficiency of MS causes metabolic changes in the neurons which subsequently decreases DNA and histone methylation reactions and promotes neuronal death (Sanchis —Segura et al., 2009; Trivedi et al., 2015).

Evidence suggests that epigenetic alterations following drug use regulate the expression of genes involved in synaptic plasticity and memory formation (Tsankova et al., 2007; Maze et al., 2011). These alterations could be responsible for the transition from experimental to compulsive drug taking. In rodent models of drug addiction (CPP and self-administration, SA), manipulation of DNA methylation, a stable epigenetic modification with inhibitors, can prevent acquisition, retrieval, and seeking behavior (Han et al., 2010; Tian et al., 2012; Dimitriu et al., 2012).

There are no FDA approved treatments for cocaine or methamphetamine (METH) use disorder (CUD/MUD) and associated neuropathy. The supportive treatments have either failed or have marginal effects. Epidemiological data suggest that CUD/MUD could be the next substance use crisis after opioids. There are no old/new pharmacotherapeutics that target the methionine synthase enzyme to prevent CUD/MUD.

### SUMMARY

The present disclosure provides thiol-containing antioxidants with therapeutic properties, pharmaceutical compositions thereof, and methods for their use.

In some aspects, the present disclosure provides compounds of the formula:

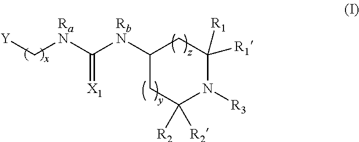

wherein:
   X₁ is O, S, or NR_{c}, wherein R_{c} is hydrogen, alkyl_{(C≤8)}, or substituted alkyl_{(C≤8)};
   R₁, R₁′, R₂, and R₂′ are each independently selected from hydrogen, alkyl_{(C≤12)}, or substituted alkyl_{(C≤12)};
   R₃ is hydroxy or an oxyl radical;
   Rₐ and R_{b} are each independently selected from hydrogen, alkyl_{(C≤6)}, or substituted alkyl_{(C≤6)};
   Y is aryl_{(C≤12)}, substituted aryl_{(C≤12)}, heteroaryl_{(C≤12)}, substituted heteroaryl_{(C≤12)}, or a group of the formula:

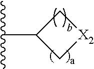

wherein:
   X₂ is O, S(O)ₘ, or NR_{d}, wherein m is 0, 1, or 2; and Rₐ is hydrogen, alkyl_{(C≤6)}, or substituted alkyl_{(C≤6)}; and
   a and b are each independently 0, 1, 2, or 3; and
x, y, and z are each independently 0, 1, 2, or 3;
or a pharmaceutically acceptable salt thereof.

In some embodiments, the compound is further defined as:

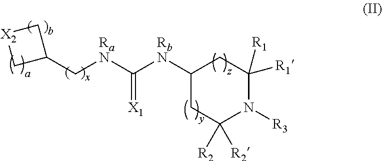

wherein:
   X₁ is O, S, or NR_{c}, wherein R_{c} is hydrogen, alkyl_{(C≤8)}, or substituted alkyl_{(C≤8)};
   R₁, R₁′, R₂, and R₂′ are each independently selected from hydrogen, alkyl_{(C≤12)}, or substituted alkyl_{(C≤12)};
   R₃ is hydroxy or an oxyl radical;
   Rₐ and R_{b} are each independently selected from hydrogen, alkyl_{(C≤6)}, or substituted alkyl_{(C≤6)};
   X₂ is O, S(O)ₘ, or NRₐ, wherein m is 0, 1, or 2; and Rₐ is hydrogen, alkyl_{(C≤6)}, or substituted alkyl_{(C≤6)};
   a and b are each independently 0, 1, 2, or 3; and
   x, y, and z are each independently 0, 1, 2, or 3;
or a pharmaceutically acceptable salt thereof.

In some embodiments, the compound is further defined as:

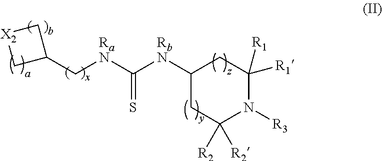

wherein:
   R₁, R₁′, R₂, and R₂′ are each independently selected from hydrogen, alkyl_{(C≤12)}, or substituted alkyl_{(C≤12)};
   R₃ is hydroxy or an oxyl radical;
   Rₐ and R_{b} are each independently selected from hydrogen, alkyl_{(C≤6)}, or substituted alkyl_{(C≤6)};
   X₂ is O, S(O)ₘ, or NRₐ, wherein m is 0, 1, or 2; and Rₐ is hydrogen, alkyl_{(C≤6)}, or substituted alkyl_{(C≤6)};
   a and b are each independently 1, 2, or 3; and
   x, y, and z are each independently 0, 1, 2, or 3;
or a pharmaceutically acceptable salt thereof.

In some embodiments, the compound is further defined as:

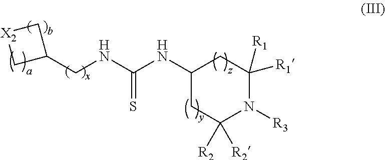

wherein:
   R₁, R₁′, R₂, and R₂′ are each independently selected from hydrogen, alkyl_{(C≤12)}, or substituted alkyl_{(C≤12)};
   R₃ is hydroxy or an oxyl radical;
   X₂ is O, S(O)ₘ, or NR_{d}, wherein m is 0, 1, or 2; and Rₐ is hydrogen, alkyl_{(C≤6)}, or substituted alkyl_{(C≤6)};
   a and b are each independently 0, 1, 2, or 3; and
   x, y, and z are each independently 0, 1, 2, or 3;
or a pharmaceutically acceptable salt thereof.

In some embodiments, the compound is further defined as:

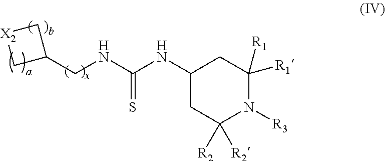

wherein:
   R₁, R₁′, R₂, and R₂′ are each independently selected from hydrogen, alkyl_{(C≤12)}, or substituted alkyl_{(C≤12)};
   R₃ is hydroxy or an oxyl radical;
   X₂ is O, S(O)ₘ, or NRₐ, wherein m is 0, 1, or 2; and Rₐ is hydrogen, alkyl_{(C≤6)}, or substituted alkyl_{(C≤6)};
   a and b are each independently 0, 1, 2, or 3; and
   z is 0, 1, 2, or 3;
or a pharmaceutically acceptable salt thereof.

In some embodiments, the compound is further defined as:

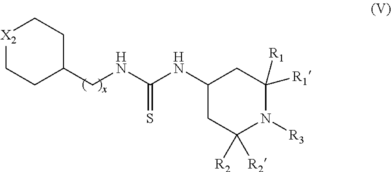

wherein:
   R₁, R₁′, R₂, and R₂′ are each independently selected from hydrogen, alkyl_{(C≤12)}, or substituted alkyl_{(C≤12)};
   R₃ is hydroxy or an oxyl radical;
   X₂ is O, S(O)ₘ, or NR_{d}, wherein m is 0, 1, or 2; and Rₐ is hydrogen, alkyl_{(C≤6)}, or substituted alkyl_{(C≤6)}; and
   x is 0, 1, 2, or 3;
or a pharmaceutically acceptable salt thereof.

In some embodiments, the compound is further defined as:

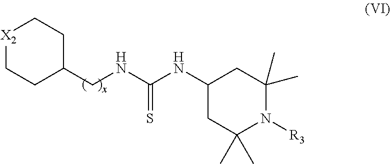

wherein:
   R₃ is hydroxy or an oxyl radical;
   X₂ is O, S(O)ₘ, or NR_{d}, wherein m is 0, 1, or 2; and Rₐ is hydrogen, alkyl_{(C≤6)}, or substituted alkyl_{(C≤6)}; and
   x is 0, 1, 2, or 3;
or a pharmaceutically acceptable salt thereof.

In some embodiments, the compound is further defined as:

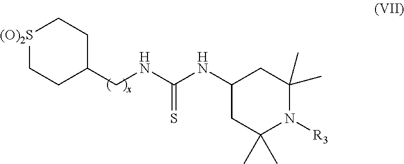

wherein:
   R₃ is hydroxy or an oxyl radical; and
   x is 0, 1, 2, or 3;
or a pharmaceutically acceptable salt thereof.

In some embodiments, Y is aryl_{(C≤12)} or substituted aryl_{(C≤12)} such as heteroaryl_{(C≤12)} or substituted heteroaryl_{(C≤12)}. In some embodiments, Y is

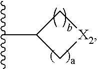

wherein: X₂ is O, S(O)ₘ, or NRₐ, wherein m is 0, 1, or 2; and Rₐ is hydrogen, alkyl_{(C≤6)}, or substituted alkyl_{(C≤6)}; and a and b are each independently 0, 1, 2, or 3. In some embodiments, X₁ is S. In other embodiments, X₁ is O. In certain embodiments, X₁ is NR_{c}. In some embodiments, Rₐ is hydrogen. In some embodiments, R_{b} is hydrogen. In some embodiments, y is 0, 1, or 2. In certain embodiments, y is 1. In some embodiments, z is 0, 1, or 2. In certain embodiments, z is 1. In some embodiments,
a is 1, 2, or 3. In certain embodiments, a is 2. In some embodiments, b is 1, 2, or 3. In certain embodiments, b is 2.

In some embodiments, R₁ is alkyl_{(C≤12)} such as methyl. In some embodiments, R₁′ is alkyl_{(C≤12)} such as methyl. In some embodiments, R₂ is alkyl_{(C≤12)}, such as methyl. In some embodiments, R₂′ is alkyl_{(C≤12)} such as methyl.

In some embodiments, X₂ is S(O)ₘ, wherein m is 0, 1, or 2. In certain embodiments, m is 2. In certain embodiments, m is 0. In some embodiments, R₃ is an oxyl radical. In some embodiments, x is 0, 1, or 2. In certain embodiments, x is 0.

In some embodiments, the compound is further defined as:

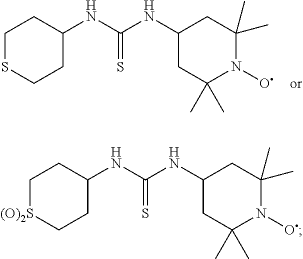

or a pharmaceutically acceptable salt thereof.

In some embodiments, the compound is further defined as:

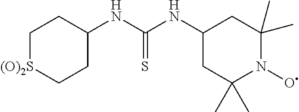

or a pharmaceutically acceptable salt thereof.

In another aspect, the present disclosure provides a pharmaceutical composition comprising:
a) a compound disclosed herein; and
b) an excipient and/or a pharmaceutically acceptable carrier.

In some embodiments, the pharmaceutical composition is formulated for administration. In some embodiments, the pharmaceutical composition is formulated as a unit dose. In another aspect, the present disclosure provides a method of treating a disease or disorder in a patient in need thereof comprising administering to the patient a therapeutically effective amount of a compound or a composition disclosed herein.

In some embodiments, the disease or disorder is a neurodegenerative disease. In some embodiments, the neurodegenerative disease is related to oxidative stress. In some embodiments, the neurodegenerative disease is related to hypoxic neural death. In some embodiments, the neurodegenerative disease is optic neuropathy. In some embodiments, the neurodegenerative disease is an age-related disease, such as Alzheimer's disease or Parkinson's disease.

In some embodiments, the disease or disorder is an addiction. In some embodiments, the addiction a drug, such as methamphetamine. In some embodiments, the drug is heroin. In some embodiments, the drug is cocaine.

In some embodiments, the patient is a mammal, such as a human. In some aspects, the method further comprises administering one or more drugs in combination with the compound or composition. In certain aspects, the method comprises administering the compound or composition once. In other aspects, the method comprises administering the compound or composition two or more times. In some embodiments, the compound or composition is administered for a period of months. In some embodiments, the compound or composition is administered indefinitely.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows cocaine or methamphetamine-induced intracellular changes leading to drug-seeking behavior.

FIG. 2 shows the energy minimized conformation of N—CH₃ coordinated SA-31 and N5-CH₃ pteridine ring of THE shows similar mode of N—C(═O/S)—N—CH₃ conformation (shown inside peach square). Binding of —CH₃ group to Nis critical and can be disrupted during oxidative stress condition. THF transfers the coordinated —CH₃ to homocysteine after attaching with B₁₂-cobalamin complex in the MS enzyme Met-H to synthesize methionine. ChemDraw 3D, V16.0

FIGS. 3A & 3B show hybrid ROS scavengers with BBB and GI permeant activities.

FIG. 4 shows a synthetic scheme of two representative compounds: SA-30 and SA-31.

FIG. 5A-5C show Compounds SA-30 and SA-31 protects neural cells from t-butyl hydrogen peroxide (TBHP), cocaine and methamphetamine induced oxidative stress and cell death in mouse hippocampal neural HT22 cells (FIG. 5A), and in human neuroblastoma SHSY5Y neuroblastoma cells (FIGS. 5B-5C). N=3. One-way ANOVA, GraphPad Prism.

FIGS. 6A-6C show cells seeded in a 12-well plate and treated with vehicle or TBHP or cocaine or METH (with or without SA analogs). Cell lysates are used to quantify MS activities using ELISA (n=3-4 technical replicates) and data are normalized to protein concentrations. *p<0.05, **p<0.01, **p<0.001 and ****p<0.0001.

FIGS. 7A-7E show that compound SA-31 increased the cocaine and METH induced decreased in SOD activity in SHSY5Y neural cells at 100 μM concentration after 24 h of treatment (FIGS. 7A & 7B). Total antioxidant concentration significantly increased after cocaine treatment (FIG. 7C) possibly activation of compensation mechanism after oxidative stress. GPX activity (FIG. 7D) was significantly increased in SA-31+cocaine treated cells. No significant changes observed in the GPX activity in cocaine+SA-31 treated cells (FIG. 7E). N=3. *p<0.05, ***p<0.001. One-way ANOVA, GraphPad Prism.

FIGS. 8A-B shows compound SA-31 decreases inflammatory cytokine IL-1ß in human SH-SY5Y neural cells.

FIGS. 9A & 9B show that compound SA-31 increases both mitochondrial respiration (FIG. 9A) and glycolytic activity (FIG. 9B) in SHSY5Y cells as compared to untreated control. N=3-4 technical replicates.

FIGS. 10A-10F show compound SA-31 is bioavailable in mouse brain after systemic 5 dosing.

FIGS. 11A & 11B show locomotor activity during expression test. Each bar represents average horizontal activity (counts)+SEM for the last 15 min of expression test. * Represents significant difference from the Unpaired-vehicle group, and † represents significant difference from the Paired-vehicle group, p<0.05.

### DESCRIPTION OF ILLUSTRATIVE EMBODIMENTS

In some aspects, the present disclosure provides in-house developed novel small thiourea containing molecules effective in mitigating cocaine or METH-seeking behavior. In other aspects, the present disclosure provides an innovative approach to the treatment of other neurodegenerative diseases resulting from oxidative stress (OS) or hypoxic neural death (ischemic stroke) including optic neuropathy and aged related diseases such as Alzheimer's disease, Parkinson's disease, etc.

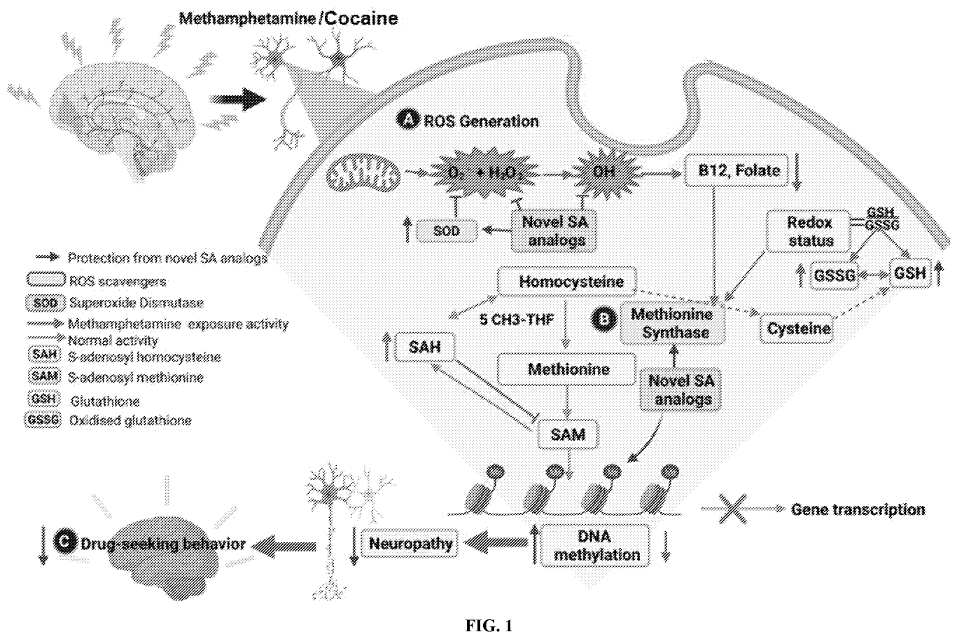

METH exposure produces high levels of OS in the mitochondria via generation of ROS. High levels of OS render the enzyme methionine synthase (MS) inactive (Hondorp et al., 2004), leading to hypomethylation of DNA and neuropathy (FIG. 1). Superoxide dismutase (SOD) reduces ROS. A SOD mimetic drug, Tempol, was also effective in reducing drug-seeking behavior in rodents (Numa et al., 2008). The novel small molecules of the present disclosure (SA analogs) have superoxide scavenging properties that mimic activities of Tempol as well as those of a known hydroxyl radical scavenger, dimethyl thiourea (DMTU) (Koblin et al., 1990). In a glaucomatous optic neuropathy mouse model, previously reported SA analogs have been reported to have extensive scavenging properties (Amankwa et al., 2021). Without wishing to be bound by any theory, it is believed that the compounds of the present disclosure which serve as super ROS scavengers, increase activity of MS, maintain the redox environment and prevent drug-seeking behavior, providing neuroprotection.

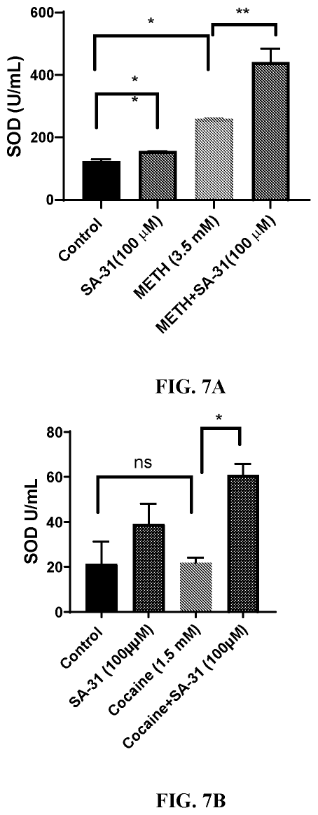

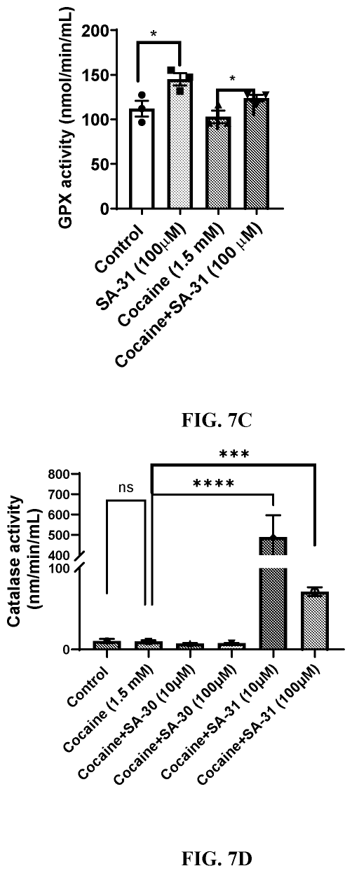

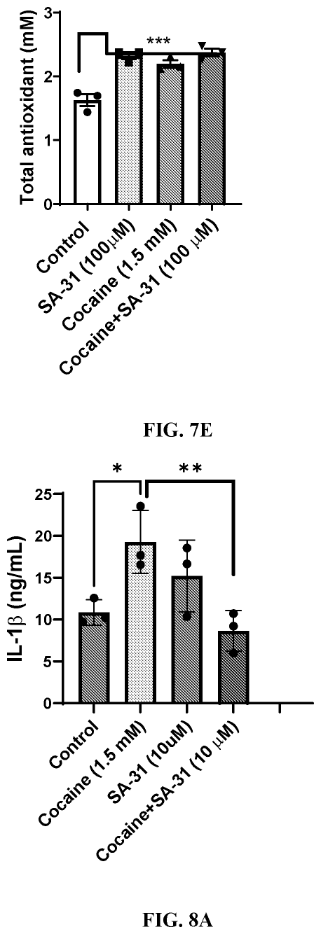

DMTU is a cell permeable peroxyl radical scavenger and MS enzyme activator activity in the brain. Increase in ROS level causes decrease in MS activity, that leads to dysregulation of DNA methylation, resulting in metabolic changes that may contribute to neural cell death (Sanchis —Segura et al., 2009; Trivedi et al., 2015; Sanchez-Gongora et al., 1997). Therefore, by scavenging both inter and intracellular ROS(O₂⁻, ·OH) from cocaine exposure, it may be possible to protect the neural cells from death. DMTU (both in 5 mg/kg and 10 mg/kg) demonstrated potent bioactivity in the mouse model of CPP (FIGS. 7A-7E). However, DMTU with very low molecular weight (<160) violates the drug-like criteria and lead-like criteria. Additionally, DMTU is not a brain blood barrier (BBB) permeable compound as predicted by Swiss ADME database. The attenuation of drug-seeking behavior by DMTU could be attributed to passive paracellular diffusion of DMTU through blood brain barrier (BBB), or to an indirect mechanism by modulating PNS.

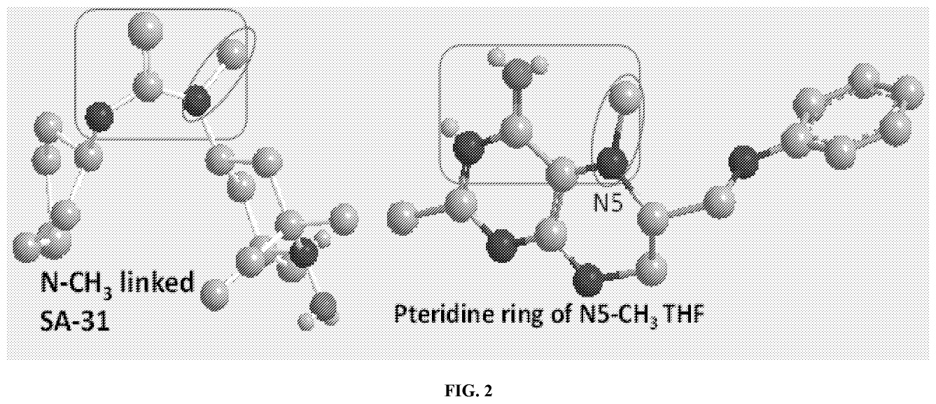

As discussed earlier, increased ROS via psychostimulant exposure could lead to DNA hypomethylation, resulting in metabolic changes that may contribute to neural cell death. The methylation reactions in the brain are regulated by MS activity, and this is achieved by disabling Vit B₁₂-folate complex mediated-CH₃ transfer to regenerate methionine (Trivedi et al., 2015). The present disclosure provides a series of novel small molecules that had dual bioactivity, first by (i) increasing intracellular MS enzyme levels in vitro possibly by mimicking the N5-CH₃ binding site of pteridine-fragment of tetrahydrofolate (THF) (FIG. 2); second by (ii) adding functional groups such as sulfoxides and nitroxides to scavenge broad spectrum ROS. Several SA analogs have been designed and synthesized. The effectiveness of two such analogs SA-30 and SA-31 was validated in protecting neural cells (HT-22 and SH-SY5Y) against OS (t-butyl hydrogen peroxide; TBHP) and toxic psychostimulant cocaine or METH insult. In a-proof-of-concept study, both SA-30 and SA-31 were shown to be good candidates for such neuroprotection, not only in an in vitro but also in an in vivo models.

Inactivation of the MS enzyme in mice (Koblin et al., 1981) and humans (Koblin et al., 1982) was caused by increased peroxyl radical (OH) production which ultimately led to dysregulation in DNA methylation machinery. The present disclosure takes an innovative approach for drug discovery and is proposing MS as a biological target for CUD/MUD. The ability of these compounds to activate MS in an in vitro model (human neural cells) was used as a screening tool. Without wishing to be bound by any particular theory, it is believed that a robust antioxidant agent that effectively scavenges both 02″ and OH radicals and upregulates MS activity will selectively reduce the retrieval of drug-associated memory. Therefore, this dual action constitutes a novel target for drug development in prevention of compulsive drug-seeking and relapse in patients with SUD.

### I. CUD Treatment

Cannabis use is associated with comorbid mental health problems, such as mood and anxiety disorders, and discontinuing cannabis use is difficult for some users. Psychiatric comorbidities are often present in dependent cannabis users including a range of personality disorders. Based on annual survey data, some high school seniors who report smoking daily (nearly 7%, according to one study) may function at a lower rate in school than students that do not (Robertson, 2018). The sedating and anxiolytic properties of tetrahydrocannbinol (THC) in some users might make the use of cannabis an attempt to self-medicate personality or psychiatric disorders.

Prolonged cannabis use produces both pharmokinetic changes (how the drug is absorbed, distributed, metabolized, and excreted) and pharmacodynamic changes (how the drug interacts with target cells) to the body. These changes require the user to consume higher doses of the drug to achieve a common desirable effect (known as a higher tolerance), reinforcing the body's metabolic systems for eliminating the drug more efficiently and further down-regulating cannabinoid receptors in the brain. Cannabis users have shown decreased reactivity to dopamine, suggesting a possible link to a dampening of the reward system of the brain and an increase in negative emotion and addiction severity (Madras, 2014).

Cannabis users can develop tolerance to the effects of THC. Tolerance to the behavioral and psychological effects of THC has been demonstrated in adolescent humans and animals. The mechanisms that create this tolerance to THC are thought to involve changes in cannabinoid receptor function. One study has shown that between 2001-2002 and 2012-2013, the use of marijuana in the US doubled. Cannabis dependence develops in about 9% of users, significantly less than that of heroin, cocaine, alcohol, and prescribed anxiolytics, but slightly higher than that for psilocybin, mescaline, or LSD (Budney et al., 20007). Of those who use cannabis daily, 10-20% develop dependence (Borgelt et al., 2013).

Cannabis withdrawal symptoms occur in half of people being treated for cannabis use disorders. Symptoms may include dysphoria, anxiety, irritability, depression, restlessness, disturbed sleep, gastrointestinal symptoms, and decreased appetite. It is often paired with rhythmic movement disorder. Most symptoms begin during the first week of abstinence and resolve after a few weeks. About 12% of heavy cannabis users showed cannabis withdrawal as defined by the DSM-5, and this was associated with significant disability as well as mood, anxiety and personality disorders.

A Cochrane review of 17 clinical studies suggested that participants treated with DA agonists like amantadine, bromocriptine, and pergolide failed to abstain from cocaine use (Minozzi, et al., 2015). Stimulant prescription drugs are thought to be a proxy for the reinforcing effects of cocaine. The prescription stimulants, methylphenidate and Damphetamine salts, dose-dependently substituted for cocaine in animal models (Rush et al., 2012). However, clinical trials with these drugs have indicated inconsistent results. Additionally, concerns of abuse liability with the stimulants themselves have added to the reluctance in prescribing these compounds for treatment of CUD. Modafinil, a drug approved for narcolepsy, inhibits DA reuptake transporter (DAT) and norepinephrine transporter (NET) (Volkow et al., 2009). Pretreatment with modafinil reduced the euphoria associated with cocaine. However, males treated with modafinil 200 mg/kg were more likely to stay abstinent than their female counterparts. In another trial, patients treated with either L-dopa/carbidopa 800/200 mg/day or naltrexone 50 mg/day remained abstinent from cocaine use, but modafinil-treated participants failed to do so.

Topiramate is an FDA-approved medication for epilepsy and migraine headaches. Early pilot studies with topiramate suggested that it can reduce cocaine craving intensity in 25% of patients. Subsequent double-blind, placebo-controlled randomized trials concluded that participants on topiramate were more likely to abstain from cocaine use and stay cocaine-free for 3 weeks (Siniscalchi et al., 2015; Kampman et al., 2013). However, this drug was ineffective in preventing cocaine dependence in patients with opioid use disorder (OUD). Vigabatrin is another FDA approved antiepileptic drug. In vivo microdialysis of low doses of vigabatrin in a rat decreased cocaine-induced increase of DA in the nucleus accumbens (NAc) and prevent cue-induced reinstatement with cocaine. However, clinical trials with vigabatrin as a treatment for CUD has had mixed results. One study reported that cocaine-dependent individuals on vigabatrin were more likely to achieve abstinence, whereas another study reported patient failure to remain abstinent from cocaine use.

Disulfiram is a copper chelator that acts on multiple enzymes in the body. Although this drug has been an effective medication during recovery from alcohol addiction, the data supporting the use of disulfiram in the treatment of CUD are not very favorable. Low doses of disulfiram were beneficial initially but failed over time as participants continued cocaine usage. Recent clinical studies in disulfiram point toward pharmacogenomics as an effective treatment strategy for tailored therapeutics in addictive individuals. Data from clinical studies indicate that participants with DβH, ANKK1, MTHFR, or DRB2 genotypes were associated with responsiveness to disulfiram pharmacotherapies for cocaine addiction (Spellicy et al., 2017). Therefore, this approach of tailored treatment strategies with genetics is a promising new area for CUD. Such a tailored treatment is Nepicastat, a selective DβH inhibitor drug. Treatment with this drug significantly reduced cocaine seeking and relapse behaviors in a rat model of cocaine self-administration. Pilot study reported significant reduction in the subjective rewarding effects of cocaine (high, good feeling). A Phase II, double-blind, placebo-controlled trial is still in progress (NCT01704196).

TA-CD active vaccine was created by combining norcocaine (metabolite of cocaine) with inactivated cholera toxin. In phase 2 trials, the results were promising as it significantly reduced cocaine intake in participants. However, a larger phase 3 trial did not mirror the same success. Based on the same theory, that the toxin attached to cocaine would stimulate production of antibodies, a cocaine hapten coupled with an adenovirus has shown promising results in preclinical trials. FDA has approved this formulation for a phase 1 trial.

There are currently 26 active trials being conducted to understand either CUD and/or look at treatment strategies to prevent cocaine abuse or relapse from abstinence (clinicaltrials.gov). Sixteen trials (out of the 26) use medication as an intervention for treatment. The medications to treat dependence and addiction to cocaine include buprenorphine, methylphenidate, amphetamine, mirtazapine, N-acetyl cysteine (NAC), lorceaserin, clavulanic acid, oxytocin, and cannabidiol. The three NAC trials and one 5-HT2CR trial are in line with the thinking of the proposed project and will be discussed below. NAC, an antioxidant, has been implicated in preclinical studies as a potential therapeutic for the prevention of relapse to cocaine and been shown to reduce cocaine dependency in human subjects as well as alter behavioral properties in rodent models. The goals of the two NAC imaging trials (NCT02994875 and NCT02124941) are to understand the neuronal networks affected by cocaine dependence and to determine if treatment with NAC alters these networks (specifically, glutamate-glutamine cycling). In the third study, investigators will examine whether 3-4-week treatment with 1200 mg BID dosing of NAC will prevent relapse with cocaine dependence (NCT03423667).

Another area of research that shows promise in explaining drug-seeking phenotypic behavior is that of stable epigenetic modifications to the DNA. DNA methylation is influenced by environmental factors and genetic variation which can modulate gene expression, thereby creating a neurological basis for addictive behavior. In genetic studies, single nucleotide polymorphism (SNP) is associated with common disease states. These SNPs are also responsible for creating or destroying the methylation sites on the DNA. Molecular interactions between two types of serotonin receptors (5-HT2CR and 5-HT2AR) may be a risk factor associated with cocaine-dependence and relapse. In order to determine the significance of this interaction, a clinical trial is in progress to compare and contrast the brain and behavior responses to impulsive action and cue reactivity in cocaine dependent participants. These participants will either have high or low functionally-relevant SNP of the serotonin receptor, 5-HT2CR (Cys23Ser) (NCT0392115).

### II. Compounds of the Present Disclosure

The compounds of the present disclosure (also referred to as “compounds disclosed herein”) are shown, for example, above, in the summary section, the Examples below, Table 1, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

TABLE 1
Compounds of the Present Disclosure

| Compound ID | Structure |
|---|---|
| SA-28 | 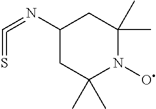 |
| SA-28A | 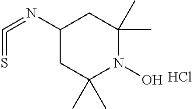 |
| SA-29 | 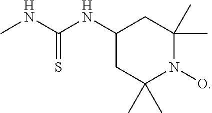 |
| SA-29A | 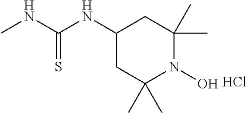 |
| SA-30-NCS | 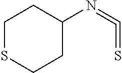 |
| SA-30-IMD | 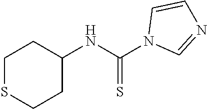 [SMILES: S=C(NC1CCSCC1)n1ccnc1] |
| SA-30 | 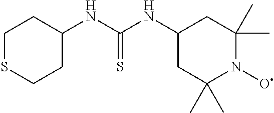 |
| SA-30A | 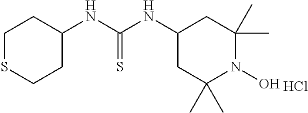 [SMILES: CC1(C)CC(NC(=S)NC2CCSCC2)CC(C)(C)N1O ; Cl] |
| SA-31-NCS | 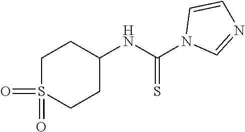 [SMILES: O=S1(=O)CCC(NC(=S)n2ccnc2)CC1] |
| SA-31 | 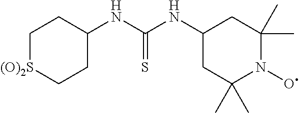 |
| SA-31A | 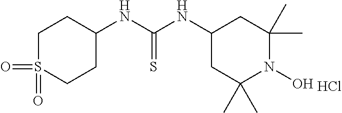 [SMILES: CC1(C)CC(NC(=S)NC2CCS(=O)(=O)CC2)CC(C)(C)N1O ; Cl] |

All the compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present disclosure have the advantage that they may be more efficacious than, be less toxic than, be longer acting than, be more potent than, produce fewer side effects than, be more easily absorbed than, more metabolically stable, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present disclosure exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs.

Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present disclosure.

### III. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22 (3): 659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the patient has eaten or will eat.

### IV. Combination Therapy

In addition to being used as a monotherapy, the compounds of the present disclosure may also find use in combination therapies. Effective combination therapy may be achieved with a single composition or pharmacological formulation that includes both agents, or with two distinct compositions or formulations, administered at the same time, wherein one composition includes a compound of this disclosure, and the other includes the second agent(s). Alternatively, the therapy may precede or follow the other agent treatment by intervals ranging from minutes to months.

Non-limiting examples of such combination therapy include combination of one or more compounds of the disclosure with another anti-inflammatory agent, a vasodilator, a ROS neutralizing agent, an antihypertensive agent, an antithrombotic agent, an agent for treating or preventing cardiovascular events such as myocardial infarction or stroke, or an analgesic agent.

### V. Definitions

The definitions below supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### A. Chemical Groups

When used in the context of a chemical group: “hydrogen” means —H; “hydroxy” means —OH; “oxo” means ═O; “oxyl radical” means —O⋅, wherein the oxygen atom has a single unpaired valence electron, and may be depicted as —O without explicit depiction of the single unpaired electron; “carbonyl” means —C(═O)—; “carboxy” means —C(—O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C—O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂; and “sulfinyl” means —S(O)—.

In the context of chemical formulas, the symbol “ [symbol] ” means a single bond, “ [symbol] ” means a double bond, and “ [symbol] ” means triple bond. The symbol “ [symbol] ” represents an optional bond, which if present is either single or double. The symbol “ [symbol] ” represents a single bond or a double bond. Thus, the formula

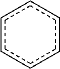

covers, for example,

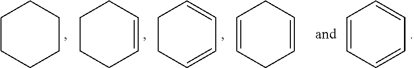

And it is understood that no one such ring atom forms part of more than one double bond. Furthermore, it is noted that the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

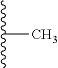

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin (cs)”, and “olefin_{C5}” are all synonymous. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C≤1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃(Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group —CRR′ in which R and R′ are independently hydrogen or alkyl. Non-limiting examples of alkylidene groups include: —CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier, one or more hydrogen atom has been independently replaced by —OH, —F, —Cl, —Br, —I, —NH₂, —NO₂, —CO₂H, —CO₂CH₃, —CN, —SH, —SCH₃, —OCH₃, —OCH₂CH₃, —C(O)CH₃, —NHCH₃, —NHCH₂CH₃, —N(CH₃)₂, C(O)NH₂, C(O)NHCH₃, C(O)N(CH₃)₂, —OC(O)CH₃, —NHC(O)CH₃, —S(O)₂OH, or —S(O)₂NH₂. The following groups are non-limiting examples of substituted alkyl groups:—CH₂OH, CH₂Cl, —CF₃, —CH₂CN, —CH₂C(O)OH, —CH₂C(O) OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

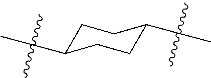 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier, one or more hydrogen atom has been independently replaced by —OH, —F, —Cl, —Br, —I, —NH₂, —NO₂, CO₂H, CO₂CH₃, —CN, —SH, —SCH₃, —OCH₃, —OCH₂CH₃, —C(O)CH₃, —NHCH₃, —NHCH₂CH₃, —N(CH₃)₂, —C(O)NH₂, —C(O)NHCH₃, C(O)N(CH₃)₂, —OC(O)CH₃, —NHC(O)CH₃, —S(O)₂OH, or —S(O)₂NH₂.

The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include:—CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, CH═C(CH₃)CH₂—, CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —SCH₃, —OCH₃, —OCH₂CH₃, —C(O)CH₃, —NHCH₃, —NHCH₂CH₃, —N(CH₃)₂, C(O)NH₂, —C(O)NHCH₃, —C(O)N(CH₃)₂, —OC(O)CH₃, —NHC(O)CH₃, S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C═CH, —C═CCH₃, and —CH₂C═CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —SCH₃, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” when used without the “substituted” modifier refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present.

If more than one ring is present, the rings may be fused or unfused. Unfused rings are connected with a covalent bond. Non-limiting examples of arenediyl groups include:

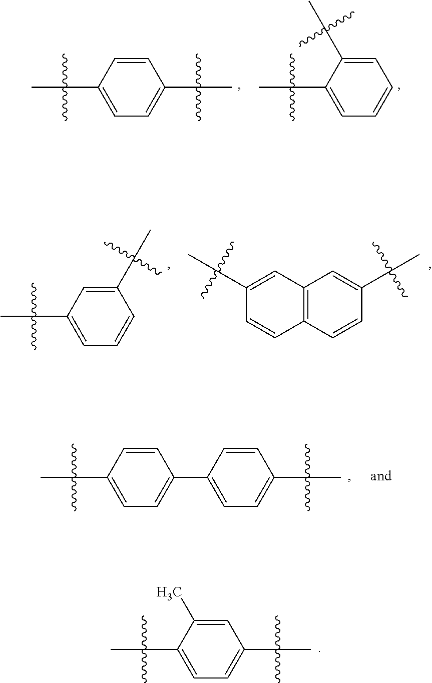

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, Br, —I, —NH₂, —NO₂, —CO₂H, —CO₂CH₃, —CN, —SH, —SCH₃, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group —alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, CO₂H, —CO₂CH₃, —CN, —SH, —SCH₃, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, CO₂H, —CO₂CH₃, —CN, —SH, —SCH₃, —OCH₃, —OCH₂CH₃, —C(O)CH₃, —NHCH₃, —NHCH₂CH₃, —N(CH₃)₂, —C(O)NH₂, C(O)NHCH₃, —C(O)N(CH₃)₂, —OC(O)CH₃, —NHC(O)CH₃, —S(O)₂OH, or —S(O)₂NH₂.

The term “heteroaralkyl” refers to the monovalent group —alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. When these terms are used with the “substituted” modifier one or more hydrogen atom has been independently replaced by —OH, —F, —Cl, —Br, —I, —NH₂, —NO₂, CO₂H, CO₂CH₃, —CN, —SH, —SCH₃, —OCH₃, —OCH₂CH₃, —C(O)CH₃, —NHCH₃, —NHCH₂CH₃, —N(CH₃)₂, —C(O)NH₂, —C(O)NHCH₃, C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, C(O) C₆H₅, and —C(O) C₆H₄CH₃ are non-limiting examples of acyl groups. A “thioacyl” is defined in an analogous manner, except that the oxygen atom of the group —C(O) R has been replaced with a sulfur atom, —C(S) R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —SCH₃, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier, one or more hydrogen atom has been independently replaced by —OH, —F, —Cl, —Br, —I, —NH₂, —NO₂, —CO₂H, —CO₂CH₃, —CN, —SH, —SCH₃, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, and “alkoxyamino” when used without the “substituted” modifier, refers to groups, defined as —NHR, in which R is cycloalkyl, alkenyl, alkynyl, aryl, aralkyl, heteroaryl, heterocycloalkyl, and alkoxy, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. When any of these terms is used with the “substituted” modifier, one or more hydrogen atom attached to a carbon atom has been independently replaced by —OH, —F, —Cl, —Br, —I, —NH₂, —NO₂, —CO₂H, —CO₂CH₃, —CN, —SH, —SCH₃, —OCH₃, —OCH₂CH₃, —C(O)CH₃, —NHCH₃, —NHCH₂CH₃, —N(CH₃)₂, —C(O)NH₂, —C(O)NHCH₃, C(O)N(CH₃)₂, —OC(O)CH₃, —NHC(O)CH₃, —S(O)₂OH, or —S(O)₂NH₂. The groups —NHC(O) OCH₃ and —NHC(O)NHCH₃ are non-limiting examples of substituted amido groups.

### B. Other Definitions

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) (also referred to as an active compound, active substance, active agent, pharmaceutical agent, agent, biologically active molecule, or a therapeutic compound) is the ingredient in a pharmaceutical drug or a pesticide that is biologically active. The similar terms active pharmaceutical ingredient (API) and bulk active are also used in medicine, and the term active substance may be used for pesticide formulations.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly (lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug) is a compound or composition used to diagnose, cure, treat, or prevent disease. An active ingredient (AI) (defined above) is the ingredient in a pharmaceutical drug or a pesticide that is biologically active. The similar terms active pharmaceutical ingredient (API) and bulk active are also used in medicine, and the term active substance may be used for pesticide formulations. Some medications and pesticide products may contain more than one active ingredient. In contrast with the active ingredients, the inactive ingredients are usually called excipients (defined above) in pharmaceutical contexts.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present disclosure. The prodrug itself may or may not also have activity with respect to a given target protein. For example, a compound comprising a hydroxy group may be administered as an ester that is converted by hydrolysis in vivo to the hydroxy compound.

Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis —β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2″, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%.

Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

As used herein, one or more of the following abbreviations may be used in the application: NO, nitric oxide; ROS, reactive oxygen species; CRAO, central retinal artery occulsion; I/R, ischemia/reperfusion; EDCI, 3-(ethyliminomethyleneamino)-N,N-dimethylpropan-1-amine, HOBt, benzotriazol-1-ol; TFA, trifluoracetic acid; DIPEA, N,N-diisopropylethylamine; and DCFDA, 2′,7′-dichlorofluorescin diacetate.

### VI. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Compounds and Synthesis

The synthesis of 4-isothiocyanato-2,2,6,6-tetramethyl-1-Piperidinyloxy (SA-28) (shown below) was achieved according to procedure outlined below:

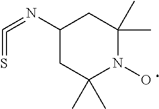

Amine (1.71 g, 10 mmol) was added to Thio-CDI (1.78 g, 10 mmol) suspension in 25 mL of THF. After overnight stirring at room temperature, the reaction mixture was concentrated to get residual oils. IN of aqueous HCl and ethyl acetate were added and was stirred for 1 hour. Solid was filtered and washed with ethyl acetate. The filtrate was washed with saturated brine solution and dried over magnesium sulfate and was filtered and concentrated to get crude solid. The crude solid was suspended in toluene and filtered to remove some undissolved dimer impurities. The filtrate is further concentrated to afford 1.94 g of pure product in 91% yield. TOF-Mass: C₁₀H₁₇N₂OS (M+H): 214.1100.

The synthesis of 4-isothiocyanato-2,2,6,6-tetramethyl-1-Piperidinyloxy HCl salt (SA-28A) (shown below) was achieved according to procedure outlined below and in the following references:
Chemistry—A European Journal, 2016, 22 (28), 9591-9598.
Bioorganic & Medicinal Chemistry Letters, 2011, 21 (1), 514-516.

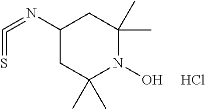

50 mg of SA-28 was dissolved in 1 mL of HCl-Methanol solution and was stirred at room temperature for 3 h. The solid formed was filtered and dried to afford 50 mg of SA-28A (white solid). ¹H NMR (300 MHz, DMSO-d₆): d 4.62-4.50 (m, 1H), 2.32-2.27 (m, 2H), 2.15-1.99 (m, 2H), 1.38 (s, 6H), 1.29 (s, 6H). TOF-Mass: C₁₀H₁₇N₂OS (M+H): 214.1100.

The synthesis of 2,2,6,6-tetramethyl-4-[[(methylamino)thioxomethyl]amino]-1-Piperidinyloxy (SA-29) (shown below) was achieved according to procedure outlined below and in the following reference:
Physical Chemistry Chemical Physics, 2016, 18 (32), 22531-22539

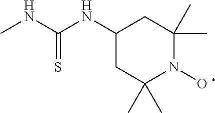

To (1.0 mmol, 214 mg) of thioisocyanate compound SA-28A in 10 mL of DCM was added methylamine (solution in THF, 31 mg, 1.0 mmol) and was stirred at room temperature for overnight. The completion of reaction was monitored by TLC and the reaction mixture was quenched with 10 ml of 1 N HCl. The mixture was extracted with DCM (2×50 mL) and the organic layer was washed with water (50 mL), brine (50 mL), and then dried over anhydrous MgSO₄. Filtration and concentration of the filtrate using a rotary evaporator gave a residue which was purified by column chromatography (SiO₂, 9:1 DCM:MeOH) to give product as an oil (220 mg, 90%). R_{f} 0.6 (7:3 EtOAc:hex). TOF-Mass: C₁₁H₂₂N₃OS (M+H): 245.1500.

The synthesis of 2,2,6,6-tetramethyl-4-[[(methylamino)thioxomethyl]amino]-1-Piperidinyloxy HCl salt (SA-29A) (shown below) was achieved according to procedure outlined below:

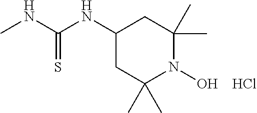

100 mg (0.409 mmol) of SA-29A was dissolved in 1 mL of HCl-Methanol solution and was stirred at room temperature for overnight. No ppt is formed. Concentrated to get 110 mg of residual oil in 97% yield (115 mg=100%).

The synthesis of 4-isothiocyanatotetrahydro-2H-thiopyran (SA-30-NCS) (shown below) was achieved according to procedure outlined below:

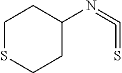

4-Amino-thiopyran (2.34 g, 20 mmol) was added to Thio-CDI (3.80 g, 10 mmol) suspension in 50 mL of DCM at 4° C. and was allowed to warm to room temperature overnight. The reaction mixture was poured into IN aq. solution, and dichloromethane and stirred for 1 hour. The undissolved solid (80 mg) was filtered and washed with dichloromethane. The filtrate was washed with saturated brine solution and dried over magnesium sulfate, filtered and was concentrated to afford 3.0 g of residual oil which was purified using column chromatography. The product was isolated by 38% EtOAc in hexane and obtained 2.1 g in 66% yields. TLC in 30% EtOAc in hexane shows product rf=0.7 and impurity is at 0.4. ¹H NMR (300 MHz, Chloroform-d): 3.89-3.81 (m 1H, —CH—N═C═S), 2.88-2.80 (m 2H, —CH₂—S—), 2.57-2.49 (m, 2H, —CH₂—S—), 2.22.-2.12 (m, 2H, —CH₂—CH₂S—), 2.09-1.99 (m, 2H, —CH₂—CH₂S—). ¹³C-NMR (75 MHz, Chloroform-d): 132.0 (N═C═S), 54.4 (—CH—N═C═S), 33.8 (—CH₂—S—), 25.1 (—CH₂—CH₂S—). FT-IR (neat) cm⁻¹:2940 C—C(s), 2072 (N═C═S), 1702 (COOH), 1428, 1358, 1288, 1185 (C-S, b). Formula: C₉H₉NS₂. Exact Mass: 159.0176. Mol. Wt: 159.2650. TOF-Mass Observed: 158.1539 (M−H).

The synthesis of N-(tetrahydro-2H-thiopyran-4-yl)-1H-imidazole-1-carbothioamide (SA-30-IMD) (shown below) was achieved according to procedure outlined below:

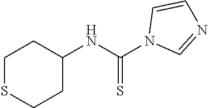 [SMILES: S=C(NC1CCSCC1)n1ccnc1]

The undissolved solid in dichloromethane upon dry obtained 80 mg in 2 yields. (4.54 g =100%). ¹H NMR (300 MHz, DMSO-d₆): 12.0 (Br-s, 1H, NH), 7.64 (s, 1H, —N═CH—N), 7.01 (s, 2H, —N—CH═CH—N), 4.09-4.01 (m 1H, —CH—N═C═S), 2.74-2.51 (m 4H, —CH₂—CH₂S—), 2.19.-2.10 (m, 2H, —CH₂—CH₂S—), 1.95-1.84 (m, 2H, —CH₂—CH₂S—). ¹³C-NMR (75 MHz, DMSO-d₆): δ 135.6 (N═C═S), 134.8 (—N═CH—N), 129.9 —N—CH═CH—N), 54.6 (—CH—N═C═S), 34.6 (—CH₂-S—), 25.0 (—CH₂—CH₂S—). FT-IR (neat) cm⁻¹:2937 C-C(s), 1557, 1427, 1358, 1211, 1114 (C-S, b). Chem. Form of compound: C₉H₁₃N₂S₂. Exact Mass: 227.2176. Mol. Wt: 227.3150. TOF-Mass Observed: 226.2164 (M−H).

The synthesis of 1-(tetrahydro-2H-thiopyran-4-yl)-3-(2,2,6,6-tetramethyl-1-(11-oxidaneyl) piperidin-4-yl)thiourea (SA30): Method-A (shown below) was achieved according to procedure outlined below:

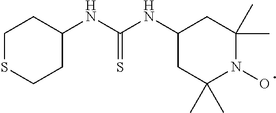

To a (1.0 mmol, 214 mg) of Thioisocyanate compounds SA-28 in 10 mL of DCM was added (117.21 mg, 1.0 mmol) of thiopyran-4-amine and was stirred at room temperature for overnight. The reaction was monitored by TLC in 50% EtOAc in hexane, once disappearance of starting material is confirmed, the reaction mixture was quenched with 10 mL of 1 N HCl. The mixture was extracted with DCM (2×50 mL) and the organic layer was washed with water (50 mL), brine (50 mL), and then dried over anhydrous MgSO₄. After filtration and concentration, the residue was purified by column chromatography (SiO₂, 9:1 DCM:MeOH) to give 310 mg of product as an pale yellow solid in 93%. R_{f} 0.4 (1:1 EtOAc: Hex). FT-IR (neat) cm⁻¹:2932 C-C(s), 1537, 1458, 1358, 1233, 1194 (C-S, b). TOF-Mass: C₁₅H₂₈N₃OS₂ (M+H): 331.1700.

The synthesis of 1-(tetrahydro-2H-thiopyran-4-yl)-3-(2,2,6,6-tetramethyl-1-(11-oxidaneyl) piperidin-4-yl)thiourea (SA30): Method-B (shown below) was achieved according to procedure outlined below:

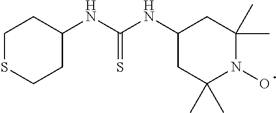 [SMILES: CC1(C)CC(NC(=S)NC2CCSCC2)CC(C)(C)N1O]

To (8.17 mmol, 1.3 g) of thioisocyanate compound SA-30—NCS in 50 mL of DCM was added (1.4 g, 8.17 mmol) of 4-Amino-tempol and was stirred at room temperature for overnight. The reaction was monitored by TLC in 50% EtOAc in hexane, once disappearance of starting material is confirmed, the reaction mixture was quenched with 10 mL of 1 N HCl.

The mixture was extracted with DCM (2×50 mL) and the organic layer was washed with water (50 mL), brine (50 mL), and then dried over anhydrous MgSO₄. After filtration and concentration, the residue was purified by column chromatography (SiO₂, 0-100% EtOAc in hexane over 15 column volumes) to give 1.4 g product as a pale yellow solid in 72.5% yield. (1.94 g, 100%).

The synthesis of 1-(tetrahydro-2H-thiopyran-4-yl)-3-(2,2,6,6-tetramethyl-1-(11-oxidaneyl) piperidin-4-yl)thiourea HCl salt (SA-30A) (shown below) was achieved according to procedure outlined below:

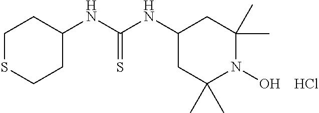 [SMILES: CC1(C)CC(NC(=S)NC2CCSCC2)CC(C)(C)N1O ; Cl]

160 mg (0.484 mmol) of SA-30 was dissolved in 1 mL of HCl-Methanol solution and was stirred at room temperature for overnight. No ppt is formed. Concentrated to get 170 mg of residual oil in 96% yield (178 mg=100%). ¹H NMR (300 MHz, Methanol-d₄): ^{□}δ 13C-NMR (75 MHz, Methanol-d₄): 1.51-1.56 (m, 14H), 1.96-2.01 (m, 3H), 21.13-2.32 (m, 5H), 2.64-2.79 (m, 4H), 4.12-4.16 (m, 1H). FT-IR (neat) cm⁻¹:2917 C-C(s), 1543, 1428, 1386, 1268, 1178 (C-S, b). Chem. Form of compound without HCl: C₁₅H₂₈N₃OS₂. Exact Mass: 330.1711. Mol. Wt: 330.5315. TOF-Mass Observed: 331.1753 (M+H).

The synthesis of N-(1,1-dioxidotetrahydro-2H-thiopyran-4-yl)-1H-imidazole-1-carbothioamide (SA-31-IMD): 4-isothiocyanatotetrahydro-2H-thiopyran (SA-31—NCS) (shown below) was achieved according to procedure outlined below:

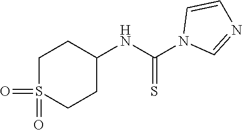 [SMILES: O=S1(=O)CCC(NC(=S)n2ccnc2)CC1]

4-Amino-thiopyran (1.5 g, 10 mmol) was added to Thio-CDI (1.85 g, 10 mmol) suspension in 50 mL of DCM at 4° C. The reaction mixture was allowed to warm to room temperature, was stirred overnight at room temperature, poured into acidic water and additional dichloromethane and was stirred for 1 hour. The undissolved solid (1.7 g) was filtered and washed with dichloromethane, washed with saturated brine solution and dried over magnesium sulfate and filtered. The filtrate upon concentration afforded 500 mg of residual oil. Crude NMR of residual oil in shows impurities which was recrystallized in acetone to provide 1.4 g of pure white solid in 54% yield (2.59 g=100%). ¹H NMR (300 MHz, DMSO-d₆): ^{□}δ 7.64 (s, 1H, —N═CH—N), 7.01 (s, 2H, —N—CH═CH—N), 4.33-4.26 (m 1H, —CH—N═C═S), 3.28-3.09 (m 4H, —CH₂—SO₂—), 2.36.-2.14 (m, 4H, (—CH₂—CH₂SO₂—). ¹³C-NMR (75 MHz, DMSO-d₆): δ 135.6 (N═C═S), 134.8 (—N═CH—N), 131.1—N—CH═CH—N), 51.9 (—CH—N═C═S), 48.9 (—CH₂—SO₂—), 30.7 (—CH₂—CH₂SO₂—). TOF-Mass: C₉H₁₃N₂O₂S₂ (M+H): 260.0532.

The synthesis of 1-(1,1-dioxidotetrahydro-2H-thiopyran-4-yl)-3-(2,2,6,6-tetramethyl-1-(11-oxidaneyl) piperidin-4-yl)thiourea (SA-31): Method-A (shown below) was achieved according to procedure outlined below:

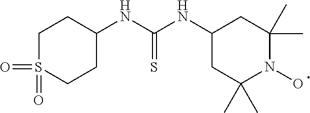

To a (1.0 mmol, 214 mg) of isothiocyanate compound SA-31—NCS in 10 mL of DCM was added (185.51 mg, 1.0 mmol) of thiopyran, 1,1, dioxide-4-amine HCl salt, and 202 mg (2.0 mmol) of TEA. The resulting mixture was stirred at room temperature for overnight and the reaction was monitored by TLC in 70% EtOAc in hexane, quenched with 10 mL of 1 N HCl. The mixture was extracted with DCM (2×50 mL) and the organic layer was washed with water (50 mL), brine (50 mL), and then dried over anhydrous MgSO₄. After filtration and concentration of the filtrate using a rotary evaporator the residue was purified by column chromatography (SiO₂, 9:1 DCM:MeOH) to give product as an oil (330 mg, 90.5%). R_{f} 0.5 (7:3 EtOAc:hex). FT-IR (neat) cm⁻¹:2974 C-C(s), 1532, 1459, 1315, 1285, 1118 (C-S, b). Chem. Form of compound: C₁₅H₂₈N₃OS₂. Exact Mass: 330. 1711.

The synthesis of 1-(1,1-dioxidotetrahydro-2H-thiopyran-4-yl)-3-(2,2,6,6-tetramethyl-1-(11-oxidaneyl) piperidin-4-yl)thiourea (SA-31): Method-B (shown below) was achieved according to procedure outlined below:

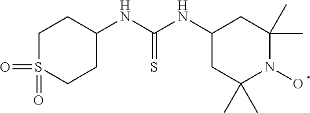

To a (1.0 g 3.86 mmol) of thiopyran, 1, 1, dioxide-4-isothiocyanate in 25 mL of THF, 4-Amino-tempol (660 mg, 3.86 mmol) was added, stirred at room temperature for overnight and was monitored by TLC in 70% EtOAc in hexane (rf 0.5). Once disappearance of starting material is confirmed, the reaction mixture was quenched with 10 mL of 1 N HCl. The mixture was extracted with DCM (2×50 mL) and the organic layer was washed with water (50 mL), brine (50 mL), and then dried over anhydrous MgSO₄. After filtration and concentration of the filtrate using a rotary evaporator the residue was purified by column chromatography (SiO₂, 0-100% EtOAc in hexane, gradient) to give product as yellow solid (900 mg, 64.5%).

The synthesis of 1-(1,1-dioxidotetrahydro-2H-thiopyran-4-yl)-3-(2,2,6,6-tetramethyl-1-(11-oxidaneyl) piperidin-4-yl)thiourea HCl salt (SA-31A) (shown below) was achieved according to procedure outlined below:

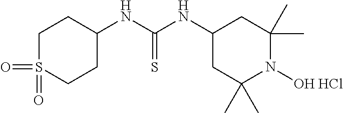

150 mg (0.414 mmol) of SA-31 was dissolved in in 1 mL of HCl-Methanol solution and was stirred at room temperature for overnight. Concentrated to get 130 mg of solid product in 93% yield. ¹H NMR (300 MHz, DMSO-d₆): ^{□} δ 11.88 (s, 1H, NH), 1185 (s, 1H, NH), 7.88-7.84 (d, 1H, NH), 4.54 (s, 1H), 4.24 (s, 1H), 3.33-3.12 (m, 4H), 2.45-2.34 (m, 4H), 2.08-1.88 (m, 4H), 1.46 (s, 6H), 1.34 (s, 6H). ¹³C-NMR (75 MHz, DMSO-d₆): δ 181.5, 67.9, 57.2, 48.8, 43.8, 30.1, 29.5, 27.7, 24.7, 20.3. FT-IR (neat) cm⁻¹:2931 C-C(s), 1538, 1315, 1282, 1119 (C-S, b). Chem. Form of compound without HCl: C₁₅H₂₉N₃OS₂. Exact Mass: 363. 1711. Mol. Wt: 363.5415. TOF-Mass Observed: 363.1753 (M+). TOF-Polar Column retention time at 3.5 minutes and peak Observed: 410.3123 (M+2H₂O+H).

### Example 2: Biological Results and Discussion

A. In vitro assay

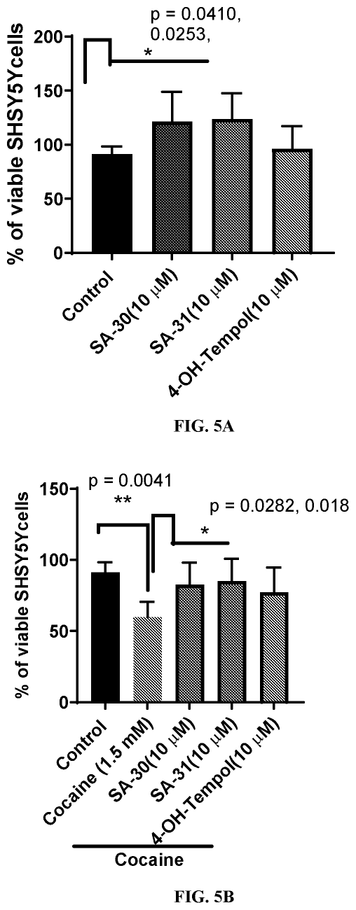

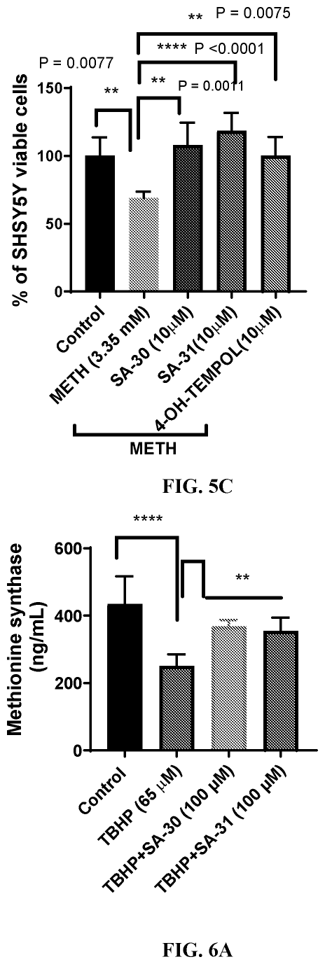

Neuroprotection activities: Either mouse hippocampal neural HT22 cells or human neuroblastoma SHSY5Y cells were cultured, seeded in 96-well plates and treated with either TBHP or cocaine or METH followed by addition of SA-30 or SA-31 at different concentrations and incubated for 24 h. MTT assay was used for measuring viable cells at absorbance of 490 nm. TBHP, cocaine or METH decreased cell proliferation to ~50% where significant cell proliferation was observed at 100 μM concentrations of SA analogs in all models. The result was compared with two known anti-oxidant compounds dimethyl thiourea (DMTU) and 4-hydoxy tempol (4-H-tempol). In both cocaine and METH induced cell death models, both SA-30 and SA-31 increased the number of viable cells compared to either DMTU or 4-H-tempol. (FIGS. 5C-5D).

### B. Compound SA-31 Increased Methionine Synthase (MS) Activity in Human SH-SY5Y Neural Cells

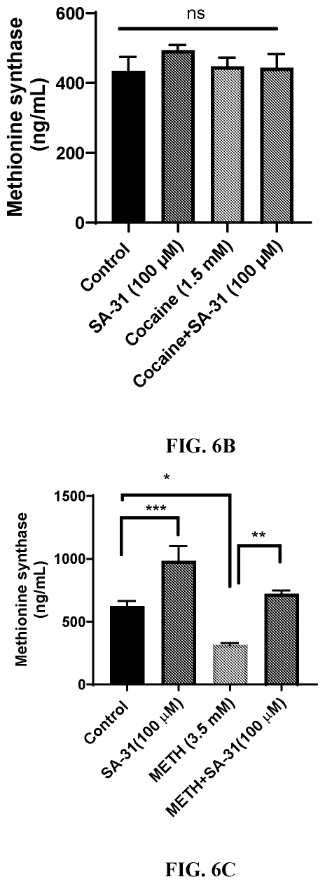

Cells were treated with either TBHP (65 μM), cocaine (1.5 mM) or METH (3.35 mM) followed by co-treatment with SA-30 and SA-31 (100 μM) for 24 hours. Cell lysates were collected and quantified with Pierce™ BCA Protein Assay. Level of intracellular methionine synthase (MS) were assessed using ELISA of the cell lysate. Both compounds SA-30 and SA-31 showed significant increase in MS activity compared to TBHP treated cells (FIG. 6A). Cocaine or cocaine_SA-31 had no significant effect on MS (FIG. 6B). However, Compound SA-31+METH group at 100 μM concentration showed significant increase in intracellular SOD and MS activity than the METH treated cells (FIG. 6C). A similar trend in TBHP experiment and SA-31 was seen at 1000 μM, demonstrating significant increase in MS enzyme.

### C. Compounds SA-30 and SA-31 Increase Activity of Antioxidant Enzymes in Human SH-SY5Y Neural Cells

As SA-31 showed improved MS activity shown in FIGS. 6A-6C, we further proceeded with SA-31 in our next experiment. Cells were treated with either cocaine (1.5 mM) or METH (3.35 mM) followed by co-treatment with SA-31 for 24 hours. Cell lysates were collected and quantified with Pierce™ BCA Protein Assay. Level of intracellular SOD, catalase, glutathione peroxidase and total antioxidant concentration were assessed using ELISA kits. Compound SA-31 showed a significant increase in SOD activity compared to either cocaine or METH cells (FIGS. 7A & B). Cocaine treatment decreased activity of glutathione peroxidase (GPx) in cells as shown in FIG. 7C while co treatment of SA-31 significantly retrieved the GPx activity. Cocaine treatment has no significant effect on catalase (CAT) activity in cells as shown in FIG. 7D while co treatment of SA-31 significantly increased the CAT activity. FIG. 7E shows that overall antioxidant concentration is increased with only SA-31 or in combination with cocaine.

### D. Compound SA-31 Decreases Inflammatory Cytokine IL-1B in Human SH-SY5Y Neural Cells

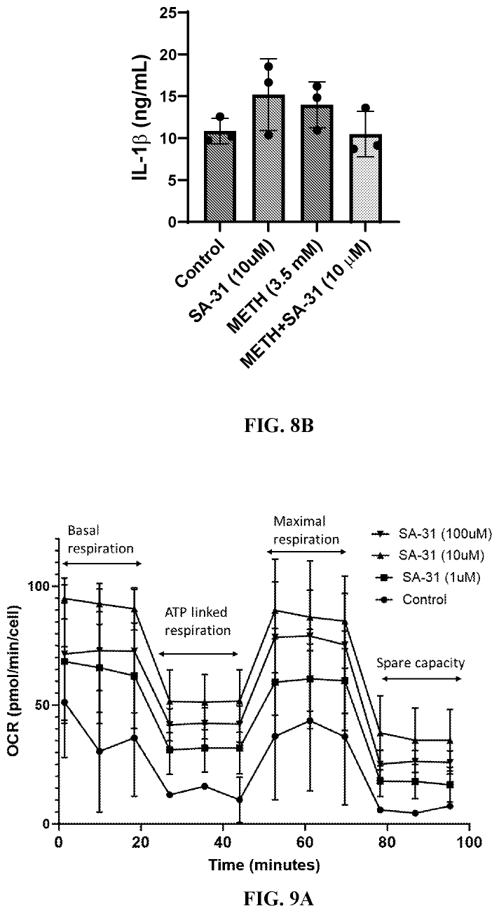

Along with neuroprotective and anti-oxidant activity, SA-31 showed anti-inflammatory activity in cells treated with cocaine or METH (FIGS. 8A-B). Briefly, SHSY-5Y cells were cultured, seeded in 96-well plates, and treated with either cocaine (1.5 mM) or MEH (3.5 mM) subsequently followed by co-treatment with SA-31 at different concentrations (10 μM, 100 μM), and incubated for 24 h. Supernatants were collected and the IL-1β cytokine concentration was assessed using ELISA kit and following manufacturer instruction (RayBiotech). Cocaine treatment significantly increased the inflammatory cytokine IL-1B while METH treatment showed a trend in increase. SA-31 (10 M) significantly decreased the cocaine induced increase in IL-1B. METH+SA-31 group showed a trend in decrease showing anti-inflammatory activity of SA-31 after both cocaine and METH insult in human brain neural cells. N=3, p<0.05; p<0.01, One-way ANOVA, GraphPad Prism.

### E. Compound SA-31 Increased Mitochondrial Bioenergetics in Human SH-SY5Y Neural Cells

In a separate experiment, 60,000 cells/well of SH-SY5Y cells were seeded in a 24 well plate and treated for 24 h with SA-31 (1 μM, 10 μM, and 100 μM). After 24 h, the mitochondrial complex inhibitors and uncoupling reagents (oligomycin, FCCP, rotenone/antimycin A) were added and the plate was analyzed for changes in oxygen consumption rate (OCR) and extracellular acidification rate (ECAR) using the Seahorse XFe24 analyzer following the manufacturer's instructions.

### F. Compound SA-31 Increases Cocaine Induced Decrease in Locomotion Activity in Mouse Model

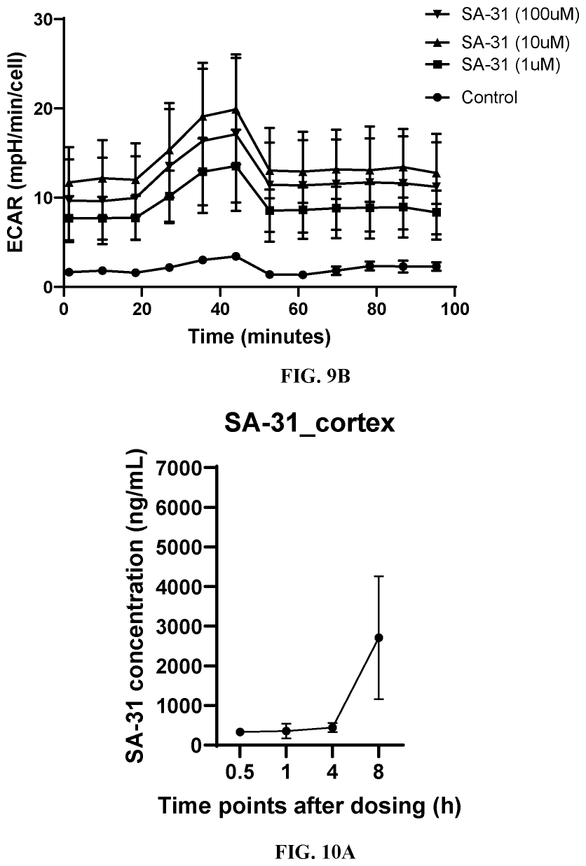

SA-31 blocks cocaine-conditioned locomotion. The goal of this pilot study was to determine if the novel small molecule was effective in preventing cocaine-conditioned motor activity of SW mice. This assay demonstrates conditioned (i.e., learned) motor activity changes following a single cocaine exposure. Cocaine results in enhanced locomotor activity in mice. Mice that were previously injected with 15 mg/kg cocaine, either in their home cages (Unpaired) or in the novel test environment (Paired), were tested 24 h later. Mice were either administered vehicle [Unpaired-vehicle or Paired-vehicle] or dimethyl thiourea (DMTU), a known hydroxyl radical scavenger or compound SA-31 prior to testing [Paired-1 mg/kg, 3 mg/kg, 5 mg/kg or 10 mg/kg]. Unpaired mice do not exhibit enhanced activity, whereas Paired mice do. However, administration of low doses of either 1 or 3 mg/kg (Paired+SA-31) prior to testing significantly inhibited expression of increased activity (FIG. 9B), whereas a higher dose of DMTU at 10 mg/kg was effective showing SA-31 is more potent than DMTU (FIG. 9A). It is yet to be determined if a high dose of SA-31 has leea to inactivation of MS in vivo by pharmacokinetic studies.

G. Compound SA-31 is Bioavailable in Mouse Brain after Systemic Dosing

The bioavailability of compound SA-31 was evaluated in adult, aged mouse (8 months old) and quantified the concentration of drug at different time points after single intraperitoneal injection. The goal was to understand whether the compound has ability to cross blood brain barrier and reach to the different region of the brain tissue systemically. Total 16 mice were used for this experiment. A single dose of SA-31 formulated in PBS was dosed intraperitoneally (10 mg/kg) to the C57Bl/6 J mice (n=4 per group, 8 months old, M+F) and were sacrificed at four different time points (0.5 h, 1 h, 4 h, and 8 h). The brain was dissected into 6 different regions (cortex, midbrain, hippocampus, cerebellum, striatum and hindbrain) and the samples were stored at −80° C. for further processing and HPLC analysis. An HPLC/LC/MS method was developed in house to quantify the concentration of SA-31 in the plasma and brain samples using Waters Atlantis HILIC Silica 3 μm, 3.0×50 mm in Agilent 1260 infinity HPLC coupled with 6460 triple Quad LC/MS. Water containing 0.1% formic acid was used as mobile phase A and acetonitrile containing 0.1% formic acid was used as mobile phase B to detect the compound and internal standard. The tissue samples were extracted, concentrated using SpeedVac and reconstituted with 50:50 isopropyl and water. The samples were filtered through 0.45-micron syringe filter and 50 μL of the sample was injected in the HLPC machine. Method validation was performed with ten serially diluted concentrations of SA-31 to generate a standard curve with >100% accuracy. The lower quantification detection range was set to be 1 ng/ml

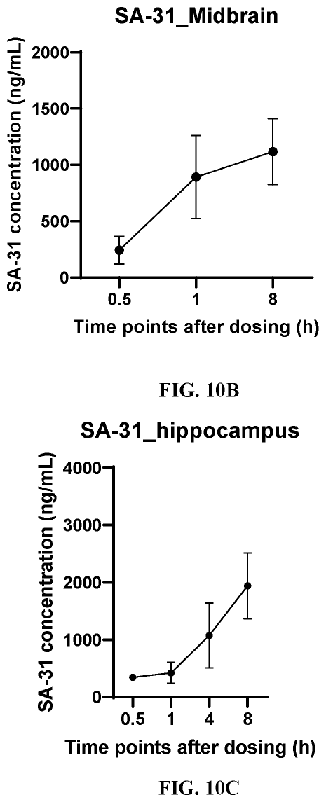

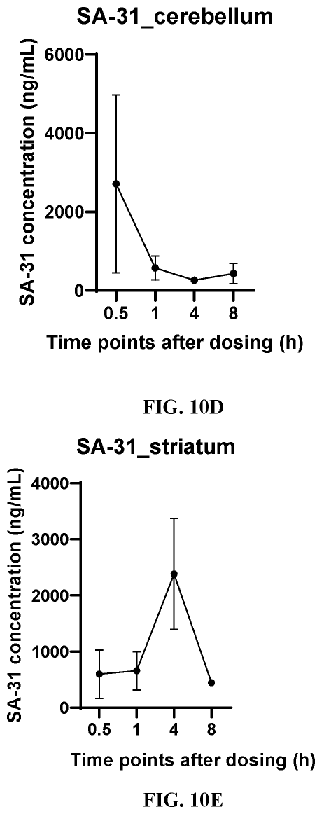

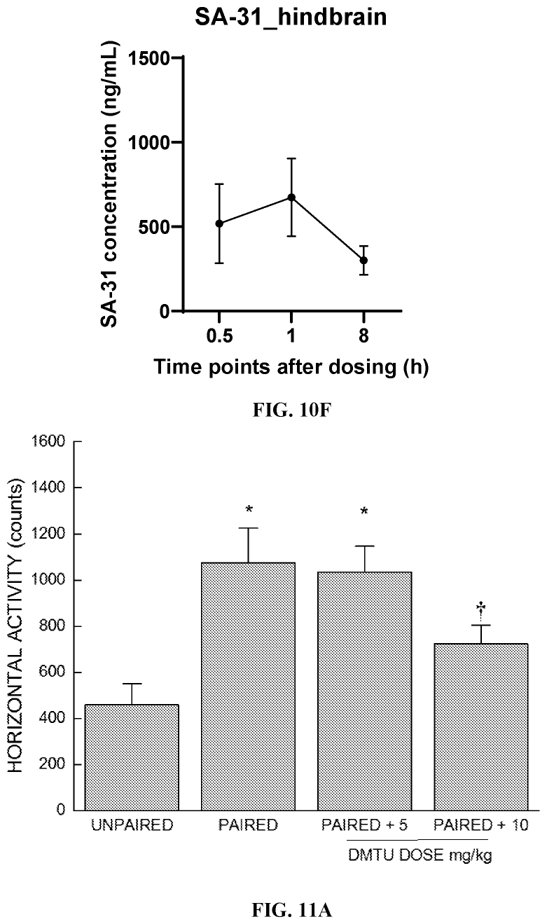

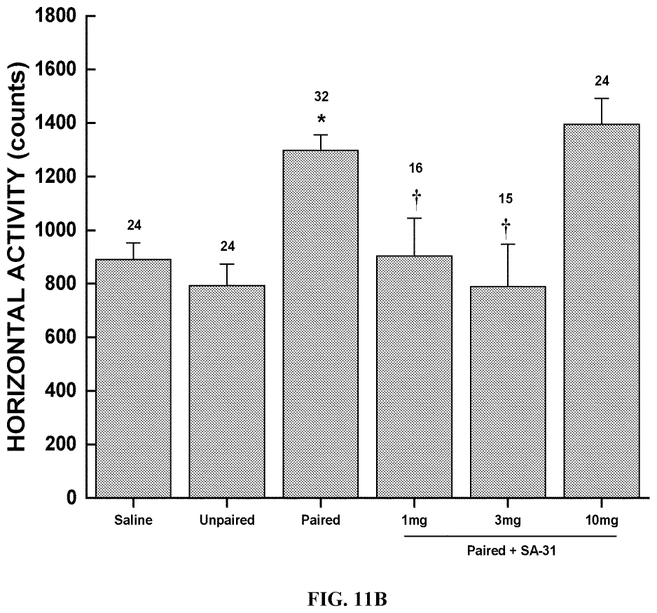

As shown in FIGS. 10A-F, compound SA-31 was successfully detected in all of the brain tissues in the range between 50 ng/mL-7500 ng/ml/per mouse brain. FIGS. 10A-C shows that, concentration of SA-31peaked at 8 h timepoint in cortex, midbrain and hippocampus respectively; FIG. 10D shows that, peak SA-31 concentration for cerebellum was achieved at 30 minutes post dosing. FIG. 10E shows that Peak SA-31 concentration for striatum was achieved at 4 h post dosing. FIG. 10F shows that Peak SA-31 concentration for hindbrain was achieved at 1 h post dosing.

### Example 3: Computational Modelling and Synthesis of Hybrid Compounds

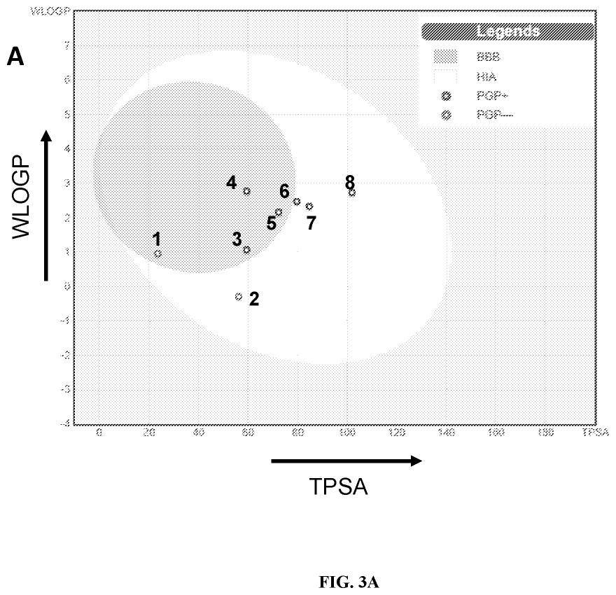

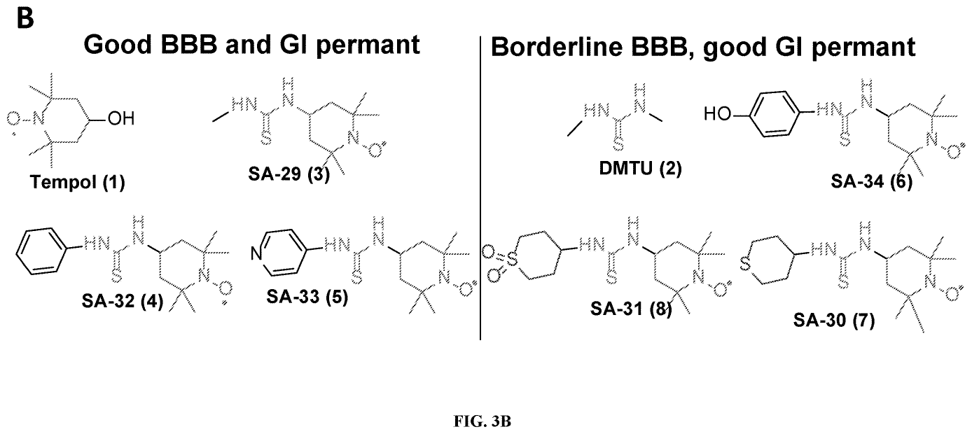

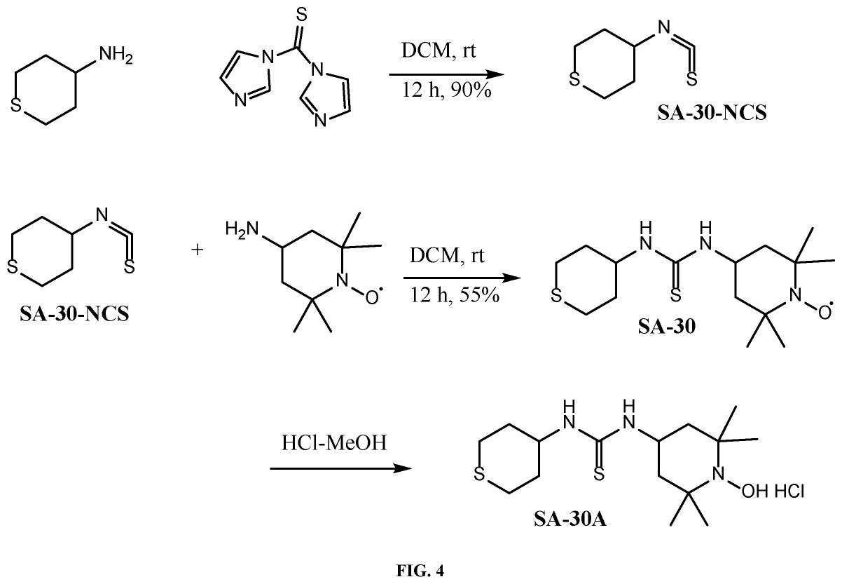

A series of hybrid antioxidants as shown in Table-1 were designed using computational modelling (SwissADME software) (Daina et al., 2017). FIG. 3A shows a plot of each compound's total polar surface area (tPSA) vs Log P (partition coefficient in octanol/H₂O) that can predict if a compound is GI permeable (will cross gastrointestinal barriers after oral or IP dosing and available systemically) or BBB permeable (available to brain) or both (FIG. 3B). The compounds that are inside the yellow oval are predicted to be BBB permeant and those are in white oval are not. The compounds with blue dots are substrates for P-glycoprotein (PGP) transporter efflux pump (responsible to efflux the drugs out from the cells) and compounds represented as red dots are not Pgp substrates. Compounds SA-30, and SA-31 have borderline but acceptable properties.

All of the compounds, formulations, and methods disclosed and claimed herein can be made and executed without undue experimentation in light of the present disclosure. While the compounds, formulations, and methods of this disclosure have been described in terms of preferred embodiments, it will be apparent to those of skill in the art that variations may be applied to the compounds, formulations, and methods, as well as in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references to the extent that they provide exemplary procedural or other details supplementary to those set forth herein, are specifically incorporated herein by reference.
Anderson, Practical Process Research & Development—A Guide for Organic Chemists, 2^{nd} ed., Academic Press, New York, 2012.
Handbook of Pharmaceutical Salts: Properties, and Use, Stahl and Wermuth Eds., Verlag Helvetica Chimica Acta, 2002.
Reagan-Shaw et al., FASEB J., 22 (3): 659-661, 2008.
Smith, March's Advanced Organic Chemistry: Reactions, Mechanisms, and Structure, 7^{th} Ed., Wiley, 2013.
Chemistry—A European Journal, 2016, 22 (28), 9591-9598.
Bioorganic & Medicinal Chemistry Letters, 2011, 21 (1), 514-516.
Physical Chemistry Chemical Physics, 2016,18 (32), 22531-22539.
Nielsen et al., Pharmacogenomics, 13 (10): 1149-1160, 2012.
Tsankova et al., Nat Rev Neurosci, 8 (5): 355-367, 2007.
Maze, et al., Ann N Y Acad Sci, 1216:99-113, 2011.
Nepal (country/area statements). Asian Pac Popul Programme News, 14 (3): 19-12, 1985.
Robertson, National Institute on Drug Abuse, 2018.
Madras, PNAS, 111 (33): 11915-11916, 2014.
Budney et al., Addiction Science and Clinical Practice, 4 (1): 4-16, 2007.
Borgelt et al., Pharmacotherapy, 33 (2): 195-209, 2013.
Han et al., Eur J Pharmacol, 642 (1-3): 93-98, 2010.
Tian et al., PLOS One, 7 (3): 33435, 2012.
Minozzi et al., Cochrane Database Syst. Rev, 2015.
Rush et al., Future Med Chem, 4 (2): 245-265, 2012.
Volkow et al., JAMA, 301 (11): 1148-1154, 2009.
Siniscalchi et al., Curr Neuropharmacol, 13 (6): 815-818, 2015.
Kampman et al., Drug Alcohol Depend, 133 (1): 94-99, 2013.
Spellicy et al., Pharmacogenet Genomics, 23 (7): 333-340, 2017.
Dumitriu et al., J Neurosci, 32 (20): 6957-6966, 2012.
Slivka et al., J Biol Chem, 260 (29): 15466-15472, 1985.
Graham D G, Mol Pharmacol, 14 (4): 633-643, 1978.
Sanchis —Segura et al., Neuropsychopharmacology, 34 (13): 2642-2654, 2009.
Trivedi et al., Nutr Metab (Lond), 12:54, 2015.
Sanchez-Gongora et al., FASEB J, 11 (12): 1013-1019, 1997.
Jones et al., MMWR Morbidity and mortality weekly report, 69:317-323, 2020.
Paulus et al., JAMA Psychiatry, 77 (9): 959-966, 2020.
Yamamoto et al., J Neuroimmune Pharmacol, 3 (4): 203-217, 2008.
Yang et al., Frontiers in molecular neuroscience, 11:186, 2018.
Kotan et al., Dusunen Adam: The Journal of Psychiatry and Neurological Sciences, 30:95-100, 2017.
Hacimusalar et al., Psychiatry and Clinical Psychopharmacology, 29 (3): 361-367, 2019.
Lin et al., RSC Advances, 9 (70): 41107-41119, 2019.
Hondorp et al., PLOS biology, 2 (11): e336, 2004.
Numa et al., Neuroscience, 155 (3): 649-658, 2008.
Beiser et al., Sci Rep, 7 (1): 11162, 2017.
Koblin et al., Br J Anaesth, 64 (2): 214-223, 1990.
Amankwa et al., Antioxidants (Basel, Switzerland, 10 (4), 2021.
Koblin et al., Anesthesiology, 54 (4): 318-324, 1981.
Koblin et al., Anesth Analg, 61 (2): 75-78, 1982.
Daina et al., Sci Rep, 7:42717, 2017.

What is claimed is:

1. A compound of the formula: 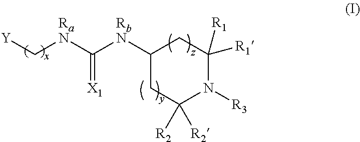
wherein:
X₁ is O, S, or NR_{c}, wherein R_{c} is hydrogen, alkyl_{(c≤8)}, or substituted alkyl_{(C≤8)};
R₁, R₁′, R₂, and R₂′ are each independently selected from hydrogen, alkyl_{(C≤12)}, or substituted alkyl_{(C≤12)};
R₃ is hydroxy or an oxyl radical;
Rₐ and R_{b} are each independently selected from hydrogen, alkyl_{(C≤6)}, or substituted alkyl_{(C≤6)};
Y is aryl_{(C≤12)}, substituted aryl_{(C≤12)}, heteroaryl_{(C≤12)}, substituted heteroaryl_{(C≤12)}, or a group of the formula: 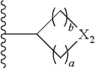
wherein:
X₂ is O, S(O)ₘ, or NRₐ, wherein m is 0, 1, or 2; and Rₐ is hydrogen, alkyl_{(C≤6)}, or substituted alkyl_{(C≤6)}; and
a and b are each independently 0, 1, 2, or 3; and
x, y, and z are each independently 0, 1, 2, or 3;
or a pharmaceutically acceptable salt thereof.

2. The compound of claim 1 further defined as: 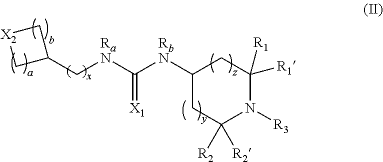
wherein:
X₁ is O, S, or NR_{c}, wherein R_{c} is hydrogen, alkyl_{(C≤8)}, or substituted alkyl_{(C≤8)};
R₁, R₁′, R₂, and R₂′ are each independently selected from hydrogen, alkyl_{(C≤12)}, or substituted alkyl_{(C≤12)};
R₃ is hydroxy or an oxyl radical;
Rₐ and R_{b} are each independently selected from hydrogen, alkyl_{(C≤6)}, or substituted alkyl_{(C≤6)};
X₂ is O, S(O)ₘ, or NR_{d}, wherein m is 0, 1, or 2; and Rₐ is hydrogen, alkyl_{(C≤6)}, or substituted alkyl_{(C≤6)};
a and b are each independently 0, 1, 2, or 3; and
x, y, and z are each independently 0, 1, 2, or 3;
or a pharmaceutically acceptable salt thereof.

3. The compound of either claim 1 or claim 2 further defined as: 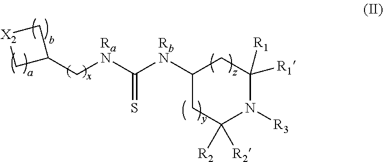
wherein:
R₁, R₁′, R₂, and R₂′ are each independently selected from hydrogen, alkyl_{(C≤12)}, or substituted alkyl_{(C≤12)};
R₃ is hydroxy or an oxyl radical;
Rₐ and R_{b} are each independently selected from hydrogen, alkyl_{(C≤6)}, or substituted alkyl_{(C≤6)};
X₂ is O, S(O)ₘ, or NR_{d}, wherein m is 0, 1, or 2; and Rₐ is hydrogen, alkyl_{(C≤6)}, or substituted alkyl_{(C≤6)};
a and b are each independently 1, 2, or 3; and
x, y, and z are each independently 0, 1, 2, or 3;
or a pharmaceutically acceptable salt thereof.

4. The compound according to any one of claims 1-3 further defined as: 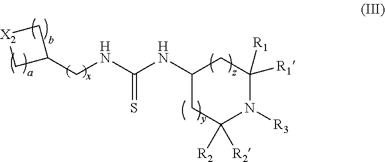
wherein:
R₁, R₁′, R₂, and R₂′ are each independently selected from hydrogen, alkyl_{(C≤12)}, or substituted alkyl_{(C≤12)};
R₃ is hydroxy or an oxyl radical;
X₂ is O, S(O)ₘ, or NR_{d}, wherein m is 0, 1, or 2; and Rₐ is hydrogen, alkyl_{(C≤6)}, or substituted alkyl_{(C≤6)};
a and b are each independently 0, 1, 2, or 3; and
x, y, and z are each independently 0, 1, 2, or 3;
or a pharmaceutically acceptable salt thereof.

5. The compound according to any one of claims 1-4 further defined as: 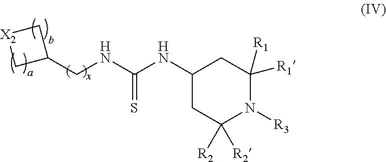
wherein:
R₁, R₁′, R₂, and R₂′ are each independently selected from hydrogen, alkyl_{(C≤12)}, or substituted alkyl_{(C≤12)};
R₃ is hydroxy or an oxyl radical;
X₂ is O, S(O)ₘ, or NRₐ, wherein m is 0, 1, or 2; and Rₐ is hydrogen, alkyl_{(C≤6)}, or substituted alkyl_{(C≤6)};
a and b are each independently 0, 1, 2, or 3; and
z is 0, 1, 2, or 3;
or a pharmaceutically acceptable salt thereof.

6. The compound according to any one of claims 1-5 further defined as: 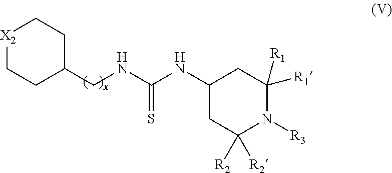
wherein:
R₁, R₁′, R₂, and R₂′ are each independently selected from hydrogen, alkyl_{(C≤12)}, or substituted alkyl_{(C≤12)};
R₃ is hydroxy or an oxyl radical;
X₂ is O, S(O)ₘ, or NRₐ, wherein m is 0, 1, or 2; and Rₐ is hydrogen, alkyl_{(C≤6)}, or substituted alkyl_{(C≤6)}; and
x is 0, 1, 2, or 3;
or a pharmaceutically acceptable salt thereof.

7. The compound according to any one of claims 1-6 further defined as: 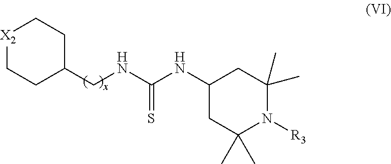
wherein:
R₃ is hydroxy or an oxyl radical;
X₂ is O, S(O)ₘ, or NR_{d}, wherein m is 0, 1, or 2; and Rₐ is hydrogen, alkyl_{(C≤6)}, or substituted alkyl_{(C≤6)}; and
x is 0, 1, 2, or 3;
or a pharmaceutically acceptable salt thereof.

8. The compound according to any one of claims 1-7 further defined as: 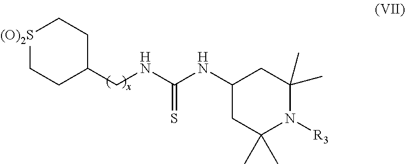
wherein:
R₃ is hydroxy or an oxyl radical; and
x is 0, 1, 2, or 3;
or a pharmaceutically acceptable salt thereof.

9. The compound of claim 1, wherein Y is aryl_{(C≤12)} or substituted aryl_{(C≤12)}.

10. The compound of claim 1, wherein Y is heteroaryl_{(C≤12)} or substituted heteroaryl_{(C≤12)}.

11. The compound of claim 1, wherein Y is 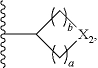
wherein: X₂ is O, S(O)ₘ, or NR_{d}, wherein m is 0, 1, or 2; and Rₐ is hydrogen, alkyl_{(C≤6)}, or substituted alkyl_{(C≤6)}; and a and b are each independently 0, 1, 2, or 3.

12. The compound according to any one of claims 1, 2, and 9-11, wherein X₁ is S.

13. The compound according to any one of claims 1, 2, and 9-11, wherein X₁ is O.

14. The compound according to any one of claims 1, 2, and 9-11, wherein X₁ is NR_{c}.

15. The compound according to any one of claims 1-3 and 9-14, wherein Rₐ is hydrogen.

16. The compound according to any one of claims 1-3 and 9-14, wherein R_{b} is hydrogen.

17. The compound according to any one of claims 1-4 and 9-16, wherein y is 0, 1, or 2.

18. The compound of claim 17, wherein y is 1.

19. The compound according to any one of claims 1-4 and 9-18, wherein z is 0, 1, or 2.

20. The compound of claim 19, wherein z is 1.

21. The compound according to any one of claims 1-5 and 9-16, wherein a is 1, 2, or 3.

22. The compound of claim 21, wherein a is 2.

23. The compound according to any one of claims 1-5 and 9-22, wherein b is 1, 2, or 3.

24. The compound of claim 23, wherein b is 2.

25. The compound according to any one of claims 1-6 and 9-24, wherein R₁ is alkyl_{(C≤12)}.

26. The compound of claim 25, wherein R₁ is methyl.

27. The compound according to any one of claims 1-6 and 9-26, wherein R₁′ is alkyl_{(C≤12)}.

28. The compound of claim 27, wherein R₁′ is methyl.

29. The compound according to any one of claims 1-6 and 9-28, wherein R₂ is alkyl_{(C≤12)}.

30. The compound of claim 29, wherein R₂ is methyl.

31. The compound according to any one of claims 1-6 and 9-30, wherein R₂′ is alkyl_{(C≤12)}.

32. The compound of claim 31, wherein R₂′ is methyl.

33. The compound according to any one of claims 1-7 and 9-32, wherein X₂ is S(O)ₘ, wherein m is 0, 1, or 2.

34. The compound according to any one of claims 1-7 and 9-33, wherein m is 2.

35. The compound according to any one of claims 1-7 and 9-33, wherein m is 0.

36. The compound according to any one of claims 1-35, wherein R₃ is an oxyl radical.

37. The compound according to any one of claims 1-36, wherein x is 0, 1, or 2.

38. The compound of claim 37, wherein x is 0.

39. The compound according to any one of claims 1-38, wherein the compound is further defined as: 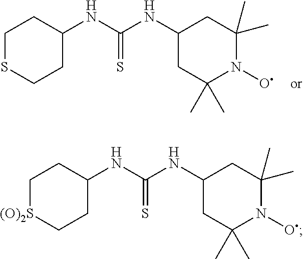
or a pharmaceutically acceptable salt thereof.

40. The compound according to any one of claims 1-39, wherein the compound is further defined as: 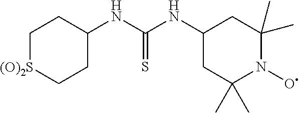
or a pharmaceutically acceptable salt thereof.

41. A pharmaceutical composition comprising:
(A) a compound according to any one of claims 1-40; and
(B) an excipient.

42. The pharmaceutical composition of claim 42, wherein the pharmaceutical composition is formulated for administration:

43. The pharmaceutical composition of either claim 41 or claim 42, wherein the pharmaceutical composition is formulated as a unit dose.

44. A method of treating a disease or disorder in a patient in need thereof comprising administering to the patient a therapeutically effective amount of a compound or a composition according to any one of claims 1-43.

45. The method of claim 44, wherein the disease or disorder is a neurodegenerative disease.

46. The method of claim 45, wherein the neurodegenerative disease is related to oxidative stress.

47. The method of claim 45, wherein the neurodegenerative disease is related to hypoxic neural death.

48. The method according to any one of claims 45-47, wherein the neurodegenerative disease is optic neuropathy.

49. The method according to any one of claims 45-47, wherein the neurodegenerative disease is an age-related disease.

50. The method of claim 49, wherein age-related disease is Alzheimer's disease or Parkinson's disease.

51. The method of claim 44, wherein the disease or disorder is an addiction.

52. The method of claim 51, wherein the addiction is a drug.

53. The method of claim 52, wherein the drug is a methamphetamine.

54. The method of claim 52, wherein the drug is heroin.

55. The method of claim 52, wherein the drug is cocaine.

56. The method according to any one of claims 44-55, wherein the patient is a mammal.

57. The method of claim 56, wherein the mammal is human.

58. The method according to any one of claims 44-57, wherein the method further comprises administering one or more drugs in combination with the compound or composition.

59. The method according to any one of claims 44-58, wherein the method comprises administering the compound or composition once.

60. The method according to any one of claims 44-58, wherein the method comprises administering the compound or composition two or more times.

61. The method of claim 60, wherein the compound or composition is administered for a period of months.

62. The method of either claim 60 or claim 61, wherein the compound or composition is administered indefinitely.
